(19) United States
(12) Patent Application Publication
(10) Pub. No.: US 2008/0070946 A1
(43) Pub. Date: Mar. 20, 2008

(54) Hydroxymethylbenzothiazoles Amides

(75) Inventors: Yevgeni Besidski, Sodertalje (SE); William Brown, Montreal (CA); Magali Harter, Rueil Malmaison (FR); Yin Hu, Sodertalje (SE); Shawn Johnstone, Montreal (CA); Paul Jones, Montreal (CA); Denis Labrecque, Montreal (CA); Alexander Munro, Montreal (CA); Christopher Walpole, Montreal (CA)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: AstraZeneca AB, Sodertalje (SE)

(21) Appl. No.: 11/576,824

(22) PCT Filed: Oct. 5, 2005
(86) PCT No.: PCT/SE05/01471

§ 371 Date: Apr. 6, 2007

(30) Foreign Application Priority Data
Oct. 8, 2004 (SE) 0402459-2
Oct. 8, 2004 (SE) 0402460-0

(51) Int. Cl. A61K 31/428; A61K 31/4436; A61K 31/4535; A61P 11/00; A61P 25/04; C07D 211/06; C07D 213/04; C07D 277/68

(52) U.S. Cl. 514/321; 514/339; 514/367; 546/198; 546/270.1; 548/178

(57) ABSTRACT

The present invention relates to new compounds, or salts, solvates or solvated salts thereof, processes for their preparation and to new intermediates used in the preparation thereof, pharmaceutical compositions containing said compounds and to the use of said compounds in therapy.

### FIELD OF THE INVENTION

The present invention relates to new compounds, to pharmaceutical compositions containing said compounds and to the use of said compounds in therapy. The present invention further relates to processes for the preparation of said compounds and to new intermediates used in the preparation thereof.

### BACKGROUND OF THE INVENTION

Pain sensation in mammals is due to the activation of the peripheral terminals of a specialized population of sensory neurons known as nociceptors. Capsaicin, the active ingredient in hot peppers, produces sustained activation of nociceptors and also produces a dose-dependent pain sensation in humans. Cloning of the vanilloid receptor 1 (VR1 or TRPV1) demonstrated that VR1 is the molecular target for capsaicin and its analogues. (Caterina, M. J., Schumacher, M. A., et. al. Nature (1997) v.389 p 816-824). Functional studies using VR1 indicate that it is also activated by noxious heat, tissue acidification) and other inflammatory mediators (Tominaga, M., Caterina, M. J. et. al. Neuron (1998) v.21, p. 531-543). Expression of VR1 is also regulated after peripheral nerve damage of the type that leads to neuropathic pain. These properties of VR1 make it a highly relevant target for pain and for diseases involving inflammation. While agonists of the VR1 receptor can act as analgesics through nociceptor destruction, the use of agonists, such as capsaicin and its analogues, is limited due to their pungency, neurotoxicity and induction of hypothermia. Instead, agents that block the activity of VR1 should prove more useful. Antagonists would maintain the analgesic properties, but avoid pungency and neurotoxicity side effects.

Compounds with VR1 inhibitor activity are believed to be of potential use for the treatment and/or prophylaxis of disorders such as pain, especially that of inflammatory or traumatic origin such as arthritis, ischaemia, cancer, fibromyalgia, low back pain and post-operative pain (Walker et al J Pharmacol Exp Ther. January 2003; 304(1): 56-62). In addition to this visceral pains such as chronic pelvic pain, cystitis, irritable bowel syndrome (IBS), pancreatitis and the like, as well as neuropathic pain such as sciatia, diabetic neuropathy, HIV neuropathy, multiple sclerosis, and the like (Walker et al ibid, Rashid et al J Pharmacol Exp Ther. March 2003; 304(3): 940-8), are potential pain states that could be treated with VR1 inhibiton. These compounds are also believed to be potentially useful for inflammatory disorders like asthma, cough, inflammatory bowel disease (IBD) (Hwang and Oh Curr Opin Pharmacol June 2002; 2(3): 235-42). Compounds with VR1 blocker activity are also useful for itch and skin diseases like psoriasis and for gastro-esophageal reflux disease (GERD), emesis, cancer, urinary incontinence and hyperactive bladder (Yiangou et al BJU Int June 2001; 87(9): 774-9, Szallasi Am J Clin Pathol (2002) 118: 110-21). VR1 inhibitors are also of potential use for the treatment and/or prophylaxis of the effects of exposure to VR1 activators like capsaicin or tear gas, acids or heat (Szallasi ibid).

A further potential use relates to the treatment of tolerance to VR1 activators.

VR1 inhibitors may also be useful in the treatment of interstitial cystitis and pain related to interstitial cystitis.

### DETAILED DESCRIPTION OF THE INVENTION

The object of the present invention is to provide compounds) exhibiting an inhibitory activity is at the vanilloid receptor 1 (VR1). The compounds of the present invention show improved stability with respect to intrinsic clearance in in-vitro in hepatocytes and in microsomes. This is generally expected to lead to an overall improvement in drugs pharmacokinetic and safety properties.

The present invention provides a compound of formula I 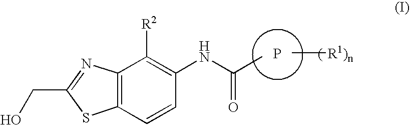
wherein:

ring P is C₆₋₁₀aryl, C₃₋₇cycloalkyl, C₅₋₆heteroaryl, which ring P may be fused with phenyl, C₅₋₆heteroaryl, C₃₋₇cycloalkyl or C₃₋₇heterocycloalkyl;

R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, OC₁₋₆haloalkyl, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆haloalkylOC₀₋₆haloalkyl, C₁₋₆alkylOC₀₋₆halolkyl, C₁₋₆alkylOC₀₋₆alkyl, OC₁₋₆alkyl, C₁₋₆alkylSC₀₋₆alkyl, C₁₋₆alkylSO, C₁₋₆alkylSO₂, C₁₋₆alkylNHCO or C₁₋₆alkylNC₀₋₆alkyl;

n is 1, 2, 3, 4 or 5; and

R² is H, F, or Cl,

or salts, solvates or solvated salts thereof.

In one embodiment the invention relates to a compound of formula I with the proviso that the compound is not

3-fluoro-N-[2-(hydroxymethyl)-1,3-benzothiazol-5-yl]-4-(trifluoromethyl)benzamide,

4-tert-butoxy-N-[4-chloro-2-(hydroxymethyl)-1,3-benzothiazol-5-yl]benzamide,

4-(tert-Butoxymethyl)-N-[4-chloro-2-(hydroxymethyl)-1,3-benzothiazol-5-yl]benzamide,

4-Bromo-2-chloro-N-[2-(hydroxymethyl)-1,3-benzothiazol-5-yl]benzamide, or

4-Bromo-2-fluoro-N-[2-(hydroxymethyl)-1,3-benzothiazol-5-yl]benzamide.

In one embodiment of the invention P is substituted with 0, 1, 2, 3 or 4 groups R¹, wherein the number of R¹ substituents on the P ring is designated by the term n. In another embodiment of the invention n is 1 or 2.

Another embodiment of the invention relates to the compound of formula I wherein ring P is phenyl.

In a further embodiment ring P is phenyl and R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, OC₁₋₆haloalkyl, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆haloalkylOC₀₋₆haloalkyl, C₁₋₆alkylOC₀₋₆halolkyl, C₁₋₆alkylOC₀₋₆alkyl, OC₁₋₆alkyl, C₁₋₆alkylSC₀₋₆alkyl, C₁₋₆alkylSO, C₁₋₆alkylSO₂ and C₁₋₆alkylNHCO or C₁₋₆alkylNC₀₋₆alkyl.

In yet another embodiment ring P is pyrazolyl, pyridine, benzdioxolane, furan, thiophene or naphthalene and R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, OC₁₋₆haloalkyl, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆haloalkylOC₀₋₆haloalkyl, C₁₋₆alkylOC₀₋₆halolkyl, C₁₋₆alkylOC₀₋₆alkyl, OC₁₋₆alkyl, C₁₋₆alkylSC₀₋₆alkyl, C₁₋₆alkylSO, C₁₋₆alkylSO₂ and C₁₋₆alkylNHCO or C₁₋₆alkylNC₀₋₆alkyl.

Ring P may be substituted by R¹ on a nitrogen or carbon atom in ring P. Further, one atom on ring P may be substituted by two substituents R¹.

In one embodiment of the invention R² is H or Cl.

In another embodiment of the invention R² is F.

In a further embodiment of the invention R¹ is selected from NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, C₁₋₆alkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl, C₁₋₆alkylSO, C₁₋₆alkylSO₂, C₁₋₆alkylNHCO, and C₁₋₆alkylNC₀₋₆alkyl.

In yet another embodiment of the invention R² is F and R¹ is selected from NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, C₁₋₆alkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl, C₁₋₆alkylSO, C₁₋₆alkylSO₂, C₁₋₆alkylNHCO, and C₁₋₆alkylNC₀₋₆alkyl.

One embodiment of the invention provides a compound of formula II

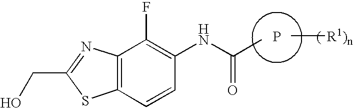

(II)

wherein:

ring P is C₆₋₁₀aryl, C₃₋₇cycloalkyl, C₅₋₆heteroaryl, which ring P may be fused with phenyl, C₅₋₆heteroaryl, C₃₋₇cycloalkyl or C₃₋₇heterocycloalkyl;

R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, C₁₋₆alkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl, C₁₋₆alkylSO, C₁₋₆alkylSO₂ and C₁₋₆alkylNHCO or C₁₋₆alkylNC₀₋₆alkyl; and

n is 1, 2, 3, 4 or 5,

or salts, solvates or solvated salts thereof.

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures.

The term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to monovalent straight or branched chain hydrocarbon radicals comprising 1 to about 12 carbon atoms.

The term “alkylene” used alone or as suffix or prefix, refers to divalent straight or branched chain hydrocarbon radicals comprising 1 to about 12 carbon atoms, which serves to links two structures together.

The term “alkenyl” used alone or as suffix or prefix, refers to a monovalent straight or branched chain hydrocarbon radical having at least one carbon-carbon double bond and comprising at least 2 up to about 12 carbon atoms.

The term “alkynyl” used alone or as suffix or prefix, refers to a monovalent straight or branched chain hydrocarbon radical having at least one carbon-carbon triple bond and comprising at least 2 up to about 12 carbon atoms.

The term “cycloalkyl,” used alone or as suffix or prefix, refers to a monovalent ring-containing hydrocarbon radical comprising at least 3 up to about 12 carbon atoms.

The term “cycloalkenyl” used alone or as suffix or prefix, refers to a monovalent ring-containing hydrocarbon radical having at least one carbon-carbon double bond and comprising at least 3 up to about 12 carbon atoms.

The term “cycloalkynyl” used alone or as suffix or prefix, refers to a monovalent ring-containing hydrocarbon radical having at least one carbon-carbon triple bond and comprising about 7 up to about 12 carbon atoms.

The term “aryl” used alone or as suffix or prefix, refers to a monovalent hydrocarbon radical having one or more polyunsaturated carbon rings having aromatic character, (e.g. 4n+2 delocalized electrons) and comprising 5 up to about 14 carbon atoms.

The term “arylene” used alone or as suffix or prefix, refers to a divalent hydrocarbon radical having one or more polyunsaturated carbon rings having aromatic character, (e.g., 4n+2 delocalized electrons) and comprising 5 up to about 14 carbon atoms, which serves to link two structures together.

The term “heterocycle” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s). Heterocycle may be saturated or unsaturated, containing one or more double bonds, and heterocycle may contain more than one ring. When a heterocycle contains more than one ring, the rings may be fused or unfused. Fused rings generally refer to at least two rings share two atoms therebetween. Heterocycle may have aromatic character or may not have aromatic character.

The term “heteroaromatic” used alone or as a suffix or prefix, refers to a ring-containing structure or molecule having one or more multivalent heteroatoms, independently selected from N, O, P and S, as a part of the ring structure and including at least 3 and up to about 20 atoms in the ring(s), wherein the ring-containing structure or molecule has an aromatic character (e.g., 4n+2 delocalized electrons).

The term “heterocyclic group,” “heterocyclic moiety,” “heterocyclic,” or “heterocyclo” used alone or as a suffix or prefix, refers to a radical derived from a heterocycle by removing one or more hydrogens therefrom.

The term “heterocyclyl” used alone or as a suffix or prefix, refers a monovalent radical derived from a heterocycle by removing one hydrogen therefrom.

The term “heterocyclylene” used alone or as a suffix or prefix, refers to a divalent radical derived from a heterocycle by removing two hydrogens therefrom, which serves to links two structures together.

The term “heteroaryl” used alone or as a suffix or prefix, refers to a heterocyclyl having aromatic character.

The term “heterocylcoalkyl” used alone or as a suffix or prefix, refers to a heterocyclyl that does not have aromatic character.

The term “heteroarylene” used alone or as a suffix or prefix, refers to a heterocyclylene having aromatic character.

The term “heterocycloalkylene” used alone or as a suffix or prefix, refers to a heterocyclylene that does not have aromatic character.

The term “six-membered” used as prefix refers to a group having a ring that contains six ring atoms.

The term “five-membered” used as prefix refers to a group having a ring that contains five ring atoms.

A five-membered ring heteroaryl is a heteroaryl with a ring having five ring atoms wherein 1, 2 or 3 ring atoms are independently selected from N, O and S.

Exemplary five-membered ring heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered ring heteroaryl is a heteroaryl with a ring having six ring atoms wherein 1, 2 or 3 ring atoms are independently selected from N, O and S.

Exemplary six-membered ring heteroaryls are pyridyl, pyrazinyl, pyrimidinyl, triazinyl and pyridazinyl.

The term “substituted” used as a prefix refers to a structure, molecule or group, wherein one or more hydrogens are replaced with one or more C₁₋₆hydrocarbon groups, or one or more chemical groups containing one or more heteroatoms selected from N, O, S, F, Cl, Br, I, and P. Exemplary chemical groups containing one or more heteroatoms include —NO₂, —OR, —Cl, —Br, —I, —F, —CF₃, —C(═O)R, —C(═O)OH, —NH₂, —SH, —NHR, —NR₂, —SR, —SO₃H, —SO₂R, —S(═O)R, —CN, —OH, —C(═O)OR, —C(═O)NR₂, —NRC(═O)R, oxo (═O), imino (═NR), thio (═S), and oximino (═N—OR), wherein each “R” is a C₁₋₆hydrocarbyl. For example, substituted phenyl may refer to nitrophenyl, methoxyphenyl, chlorophenyl, aminophenyl, etc., wherein the nitro, methoxy, chloro, and amino groups may replace any suitable hydrogen on the phenyl ring.

The term “substituted” used as a suffix of a first structure, molecule or group, followed by one or more names of chemical groups refers to a second structure, molecule or group, which is a result of replacing one or more hydrogens of the first structure, molecule or group with the one or more named chemical groups. For example, a “phenyl substituted by nitro” refers to nitrophenyl.

Heterocycle includes, for example, monocyclic heterocycles such as: aziridine, oxirane, thiirane, azetidine, oxetane, thietane, pyrrolidine, pyrroline, imidazolidine, pyrazolidine, pyrazoline, dioxolane, sulfolane 2,3-dihydrofuran, 2,5-dihydrofuran tetrahydrofuran, thiophane, piperidine, 1,2,3,6-tetrahydro-pyridine, piperazine, morpholine, thiomorpholine, pyran, thiopyran, 2,3-dihydropyran, tetrahydropyran, 1,4-dihydropyridine, 1,4-dioxane, 1,3-dioxane, dioxane, homopiperidine, 2,3,4,7-tetrahydro-1H-azepine homopiperazine, 1,3-dioxepane, 4,7-dihydro-1,3-dioxepin, and hexamethylene oxide.

In addition, heterocycle includes aromatic heterocycles, for example, pyridine, pyrazine, pyrimidine, pyridazine, thiophene, furan, furazan, pyrrole, imidazole, thiazole, oxazole, pyrazole, isothiazole, isoxazole, 1,2,3-triazole, tetrazole, 1,2,3-thiadiazole, 1,2,3-oxadiazole, 1,2,4-triazole, 1,2,4-thiadiazole, 1,2,4-oxadiazole, 1,3,4-triazole, 1,3,4-thiadiazole, and 1,3,4-oxadiazole.

Additionally, heterocycle encompass polycyclic heterocycles, for example, indole, indoline, isoindoline, quinoline, tetrahydroquinoline, isoquinoline, tetrahydroisoquinoline, 1,4-benzodioxan, coumarin, dihydrocoumarin, benzofuran, 2,3-dihydrobenzofuran, isobenzofuran, chromene, chroman, isochroman, xanthene, phenoxathiin, thianthrene, indolizine, isoindole, indazole, purine, phthalazine, naphthyridine, quinoxaline, quinazoline, cinnoline, pteridine, phenanthridine, perimidine, phenanthroline, phenazine, phenothiazine, phenoxazine, 1,2-benzisoxazole, benzothiophene, benzoxazole, benzthiazole, benzimidazole, benztriazole, thioxanthine, carbazole, carboline, acridine, pyrolizidine, and quinolizidine.

In addition to the polycyclic heterocycles described above, heterocycle includes polycyclic heterocycles wherein the ring fusion between two or more rings includes more than one bond common to both rings and more than two atoms common to both rings. Examples of such bridged heterocycles include quinuclidine, diazabicyclo[2.2.1]heptane and 7-oxabicyclo[2.2.1]heptane.

Heterocyclyl includes, for example, monocyclic heterocyclyls, such as: aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydro-pyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl.

In addition, heterocyclyl includes aromatic heterocyclyls or heteroaryl, for example, pyridinyl, pyrazinyl, pyrimidinyl, pyridazinyl, thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4 oxadiazolyl.

Additionally, heterocyclyl encompasses polycyclic heterocyclyls (including both aromatic or non-aromatic), for example, indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl, acridinyl, pyrolizidinyl, and quinolizidinyl.

In addition to the polycyclic heterocyclyls described above, heterocyclyl includes polycyclic heterocyclyls wherein the ring fusion between two or more rings includes more than one bond common to both rings and more than two atoms common to both rings. Examples of such bridged heterocycles include quinuclidinyl, diazabicyclo[2.2.1]heptyl; and 7-oxabicyclo[2.2.1]heptyl.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amine” or “amino” used alone or as a suffix or prefix, refers to radicals of the general formula —NRR′, wherein R and R′ are independently selected from hydrogen or a hydrocarbon radical.

Halogen includes fluorine, chlorine, bromine and iodine.

“Halogenated,” used as a prefix of a group, means one or more hydrogens on the group is replaced with one or more halogens.

In this specification, unless stated otherwise, the term “haloalkyl” means an alkyl group as defined above, which is substituted with halo as defined above. The term “C₁₋₆haloalkyl” may include, but is not limited to fluoromethyl, difluoromethyl, trifluoromethyl, fluoroethyl, difluoroethyl, pentafluoroethyl or bromopropyl. The term “C₁₋₆haloalkylO” may include, but is not limited to fluoromethoxy, difluoromethoxy, trifluoromethoxy, fluoroethoxy, difluoroethoxy or tri-and tetrafluoroethoxy.

“RT” or “rt” means room temperature.

Another embodiment of the invention relates to compounds selected from the group consisting

3-tert-butoxy-N-[2-(hydroxymethyl)-1,3-benzothiazol-5-yl]benzamide,

4-(dimethylamino)-N-[2-(hydroxymethyl)-1,3-benzothiazol-5-yl]benzamide,

tert-butyl 4-({[2-(hydroxymethyl)-1,3-benzothiazol-5-yl]amino}carbonyl)benzoate,

N,N-diethyl-N′-[2-(hydroxymethyl)-1,3-benzothiazol-5-yl]terephthalamide,

N-[2-(hydroxymethyl)-1,3-benzothiazol-5-yl]-4-(1,1,2,2-tetrafluoroethoxy)benzamide,

4-Cyclohexyl-N-[2-(hydroxymethyl)-1,3-benzothiazol-5-yl]benzamide,

3-Fluoro-N-[2-(hydroxymethyl)-1,3-benzothiazol-5-yl]-4-(trifluoromethyl)benzamide,

N-[2-(hydroxymethyl)-1,3-benzothiazol-5-yl]-6-(trifluoromethyl)nicotinamide,

N-[2-(hydroxymethyl)-1,3-benzothiazol-5-yl]-6-piperidin-1-ylnicotinamide,

4-(dimethylamino)-N-[2-(hydroxymethyl)-1,3-benzothiazol-5-yl]-1-naphthamide,

2-fluoro-N-[2-(hydroxymethyl)-1,3-benzothiazol-5-yl]-4-(trifluoromethyl)benzamide,

N-[2-(hydroxymethyl)-1,3-benzothiazol-5-yl]-2-methoxy-6-(trifluoromethyl)nicotinamide,

N-[2-(hydroxymethyl)-1,3-benzothiazol-5-yl]-2-methyl-6-(trifluoromethyl)nicotinamide,

3-fluoro-N-[2-(hydroxymethyl)-1,3-benzothiazol-5-yl]-2,4-dimethylbenzamide,

N-[2-(hydroxymethyl)-1,3-benzothiazol-5-yl]-2,4-dimethylbenzamide,

4-tert-butoxy-N-[4-chloro-2-(hydroxymethyl)-1,3-benzothiazol-5-yl]benzamide,

4-(tert-Butoxymethyl)-N-[4-chloro-2-(hydroxymethyl)-1,3-benzothiazol-5-yl]benzamide,

N-[4-chloro-2-(hydroxymethyl)-1,3-benzothiazol-5-yl]-4-isopropoxybenzamide,

3-tert-butoxy-N-[4-chloro-2-(hydroxymethyl)-1,3-benzothiazol-5-yl]benzamide,

tert-butyl 4-({[4-chloro-2-(hydroxymethyl)-1,3-benzothiazol-5-yl]amino}carbonyl)benzoate,

4-Bromo-2-chloro-N-[2-(hydroxymethyl)-1,3-benzothiazol-5-yl]benzamide,

4-Bromo-2-fluoro-N-[2-(hydroxymethyl)-1,3-benzothiazol-5-yl]benzamide,

4-tert-butoxy-N-[2-(hydroxymethyl)-1,3-benzothiazol-5-yl]-2-methylbenzamide,

N-[2-(hydroxymethyl)-1,3-benzothiazol-5-yl]-4-isopropoxy-2-methylbenzamide,

N-[2-(hydroxymethyl)-1,3-benzothiazol-5-yl]-4-(trifluoromethyl)benzamide,

2,3-difluoro-N-[2-(hydroxymethyl)-1,3-benzothiazol-5-yl]-4-(trifluoromethyl)benzamide,

4-fluoro-N-[2-(hydroxymethyl)-1,3-benzothiazol-5-yl]-3-(trifluoromethyl)benzamide,

4-tert-butyl-N-[2-(hydroxymethyl)-1,3-benzothiazol-5-yl]-2,6-dimethylbenzamide,

N-[2-(hydroxymethyl)-1,3-benzothiazol-5-yl]-4-methoxy-2-methylbenzamide, and

4-(difluoromethoxy)-N-[2-(hydroxymethyl)-1,3-benzothiazol-5-yl]benzamide,

or salts, solvates or solvated salts thereof; in another embodiment the invention relates to these compounds with with the proviso that the compound is not

3-fluoro-N-[2-(hydroxymethyl)-1,3-benzothiazol-5-yl]-4-(trifluoromethyl)benzamide,

4-tert-butoxy-N-[4-chloro-2-(hydroxymethyl)-1,3-benzothiazol-5-yl]benzamide,

4-(tert-Butoxymethyl)-N-[4-chloro-2-(hydroxymethyl)-1,3-benzothiazol-5-yl]benzamide,

4-Bromo-2-chloro-N-[2-(hydroxymethyl)-1,3-benzothiazol-5-yl]benzamide, or

4-Bromo-2-fluoro-N-[2-(hydroxymethyl)-1,3-benzothiazol-5-yl]benzamide.

Another embodiment of the invention relates to compounds selected from the group consisting

N-[2-(hydroxymethyl)-1,3-benzothiazol-5-yl]-2-methyl-4-(1,1,2,2-tetrafluoroethoxy)benzamide,

3-fluoro-N-[2-(hydroxymethyl)-1,3-benzothiazol-5-yl]-2-methyl-4-(trifluoromethyl)benzamide,

N-[2-(hydroxymethyl)-1,3-benzothiazol-5-yl]-2-(methoxymethyl)-4-(trifluoromethyl)benzamide,

N-[2-(hydroxymethyl)-1,3-benzothiazol-5-yl]-2-(methoxymethyl)-4-(trifluoromethoxy)benzamide,

N-[2-(hydroxymethyl)-1,3-benzothiazol-5-yl]-2-(methoxymethyl)-4-(1,1,2,2-tetrafluoroethoxy)benzamide,

3-fluoro-N-[2-(hydroxymethyl)-1,3-benzothiazol-5-yl]-2-(methoxymethyl)-4-(trifluoromethyl)benzamide,

4-bromo-N-[2-(hydroxymethyl)-1,3-benzothiazol-5-yl]-2-(methoxymethyl)benzamide,

2-(aminomethyl)-4-bromo-N-[2-(hydroxymethyl)-1,3-benzothiazol-5-yl]benzamide,

4-bromo-N-[2-(hydroxymethyl)-1,3-benzothiazol-5-yl]-2-(pyrrolidin-1-ylmethyl)benzamide,

4-bromo-N-[2-(hydroxymethyl)-1,3-benzothiazol-5-yl]-2-(piperidin-1-ylmethyl)benzamide,

4-bromo-2-({[2-(dimethylamino)ethyl]amino}methyl)-N-[2-(hydroxymethyl)-1,3-benzothiazol-5-yl]benzamide,

4-bromo-N-[2-(hydroxymethyl)-1,3-benzothiazol-5-yl]-2-(morpholin-4-ylmethyl)benzamide,

2-[(acetylamino)methyl]-4-bromo-N-[2-(hydroxymethyl)-1,3-benzothiazol-5-yl]benzamide,

4-bromo-2-{[2-(dimethylamino)ethoxy]methyl}-N-[2-(hydroxymethyl)-1,3-benzothiazol-5-yl]benzamide,

N-[2-(hydroxymethyl)-1,3-benzothiazol-5-yl]-4-isopropoxy-2-(methoxymethyl)benzamide,

4-tert-butoxy-N-[2-(hydroxymethyl)-1,3-benzothiazol-5-yl]-2-(methoxymethyl)benzamide,

{[2-({[2-(hydroxymethyl)-1,3-benzothiazol-5-yl]amino}carbonyl)-5-isopropoxybenzyl]oxy}acetic acid,

{[5-tert-butoxy-2-({[2-(hydroxymethyl)-1,3-benzothiazol-5-yl]amino}carbonyl)benzyl]oxy}acetic acid,

2-(acetylamino)-N-[2-(hydroxymethyl)-1,3-benzothiazol-5-yl]-6-(trifluoromethyl)nicotinamide,

2-{[2-(dimethylamino)ethyl]amino}-N-[2-(hydroxymethyl)-1,3-benzothiazol-5-yl]-6-(trifluoromethyl)nicotinamide,

2-amino-N-[2-(hydroxymethyl)-1,3-benzothiazol-5-yl]-6-(trifluoromethyl)nicotinamide,

2-chloro-N-[2-(hydroxymethyl)-1,3-benzothiazol-5-yl]-6-(trifluoromethyl)nicotinamide,

{[5-tert-butoxy-2-({[2-(hydroxymethyl)-1,3-benzothiazol-5-yl]amino}carbonyl)benzyl]oxy}acetic acid,

{[2-({[2-(hydroxymethyl)-1,3-benzothiazol-5-yl]amino}carbonyl)-5-isopropoxybenzyl]oxy}acetic acid,

2-hydroxy-N-[2-(hydroxymethyl)-1,3-benzothiazol-5-yl]-6-(trifluoromethyl)nicotinamide,

2-(formylamino)-N-[2-(hydroxymethyl)-1,3-benzothiazol-5-yl]-6-(trifluoromethyl)nicotinamide,

N-[2-(hydroxymethyl)-1,3-benzothiazol-5-yl]-4-methyl-2-(methylthio)pyrimidine-5-carboxamide,

4-[(3,4-dichlorobenzyl)thio]-N-[2-(hydroxymethyl)-1,3-benzothiazol-5-yl]-2-methylbenzamide,

4-[(4-chlorobenzyl)thio]-N-[2-(hydroxymethyl)-1,3-benzothiazol-5-yl]-2-methylbenzamide,

4-[(3,4-dichlorobenzyl)sulfonyl]-N-[2-(hydroxymethyl)-1,3-benzothiazol-5-yl]-2-methylbenzamide,

4-[(4-chlorobenzyl)sulfonyl]-N-[2-(hydroxymethyl)-1,3-benzothiazol-5-yl]-2-methylbenzamide,

N-[2-(hydroxymethyl)-1,3-benzothiazol-5-yl]-2-methyl-4-(methylsulfonyl)benzamide,

N-[2-(hydroxymethyl)-1,3-benzothiazol-5-yl]-2-(methoxymethyl)-4-(4-methylpiperazin-1-yl)benzamide,

N-[2-(hydroxymethyl)-1,3-benzothiazol-5-yl]-2-(methoxymethyl)-4-morpholin-4-ylbenzamide,

4-(4-acetylpiperazin-1-yl)-N-[2-(hydroxymethyl)-1,3-benzothiazol-5-yl]-2-methylbenzamide,

N-[2-(hydroxymethyl)-1,3-benzothiazol-5-yl]-2-methyl-4-piperazin-1-ylbenzamide,

N-[2-(hydroxymethyl)-1,3-benzothiazol-5-yl]-2-methyl-4-(4-methylpiperazin-1-yl)benzamide,

N-[2-(hydroxymethyl)-1,3-benzothiazol-5-yl]-2-methyl-4-morpholin-4-ylbenzamide,

N-[2-(hydroxymethyl)-1,3-benzothiazol-5-yl]-2-methyl-4-piperidin-1-ylbenzamide,

4-(dimethylamino)-N-[2-(hydroxymethyl)-1,3-benzothiazol-5-yl]-2-methylbenzamide,

2-chloro-N-[2-(hydroxymethyl)-1,3-benzothiazol-5-yl]-3,4-dimethoxybenzamide,

2-chloro-N-[2-(hydroxymethyl)-1,3-benzothiazol-5-yl]-4-isopropoxy-3-methoxybenzamide,

2-bromo-N-[2-(hydroxymethyl)-1,3-benzothiazol-5-yl]-3,4-dimethoxybenzamide,

N-[2-(hydroxymethyl)-1,3-benzothiazol-5-yl]-3,4-dimethoxy-2-methylbenzamide,

2-chloro-N-[2-(hydroxymethyl)-1,3-benzothiazol-5-yl]-4,5-dimethoxybenzamide,

2-bromo-N-[2-(hydroxymethyl)-1,3-benzothiazol-5-yl]-4,5-dimethoxybenzamide,

N-[4-fluoro-2-(hydroxymethyl)-1,3-benzothiazol-5-yl]-4-isopropoxy-2-methylbenzamide,

N-[2-(hydroxymethyl)-1,3-benzothiazol-5-yl]-6-(2,2,2-trifluoroethoxy)nicotinamide,

N-[2-(hydroxymethyl)-1,3-benzothiazol-5-yl]-1-methyl-3-(trifluoromethyl)-1H-pyrazole-5-carboxamide,

N-[2-(hydroxymethyl)-1,3-benzothiazol-5-yl]-2,4-bis(trifluoromethyl)benzamide,

2-but-3-en-1-yl-N-[2-(hydroxymethyl)-1,3-benzothiazol-5-yl]-4-methoxybenzamide,

N-[2-(hydroxymethyl)-1,3-benzothiazol-5-yl]-2-(methylamino)-6-(trifluoromethyl)-nicotinamide,

N-[2-(hydroxymethyl)-1,3-benzothiazol-5-yl]-2-(propylamino)-6-(trifluoromethyl)-nicotinamide,

2-(dimethylamino)-N-[2-(hydroxymethyl)-1,3-benzothiazol-5-yl]-6-(trifluoromethyl)-nicotinamide,

2-ethyl-N-[2-(hydroxymethyl)-1,3-benzothiazol-5-yl]-4-isopropoxybenzamide,

2-butyl-N-[2-(hydroxymethyl)-1,3-benzothiazol-5-yl]-4-methoxybenzamide,

N-[4-fluoro-2-(hydroxymethyl)-1,3-benzothiazol-5-yl]-2-methyl-6-(trifluoromethyl)nicotinamide,

N-[4-fluoro-2-(hydroxymethyl)-1,3-benzothiazol-5-yl]-6-trifluoromethyl)nicotinamide,

N-[4-fluoro-2-(hydroxymethyl)-1,3-benzothiazol-5-yl]-6-(2,2,2-trifluoroethoxy)nicotinamide,

N-[4-fluoro-2-(hydroxymethyl)-1,3-benzothiazol-5-yl]-1-methyl-3-(trifluoromethyl)-1H-pyrazole-5-carboxamide,

4-(dimethylamino)-N-[2-(hydroxymethyl)-1,3-benzothiazol-5-yl]-3,5-dinitrobenzamide,

N-[2-(hydroxymethyl)-1,3-benzothiazol-5-yl]-2-methyl-5-(trifluoromethyl)benzamide,

N-[2-(hydroxymethyl)-1,3-benzothiazol-5-yl]-5,6,7,8-tetrahydronaphthalene-2-carboxamide,

N-[2-(hydroxymethyl)-1,3-benzothiazol-5-yl]-4-vinylbenzamide,

4-ethynyl-N-[2-(hydroxymethyl)-1,3-benzothiazol-5-yl]benzamide,

4-bromo-N-[2-(hydroxymethyl)-1,3-benzothiazol-5-yl]-2-(methoxymethyl)benzamide,

1-ethyl-N-[2-(hydroxymethyl)-1,3-benzothiazol-5-yl]-1H-indole-3-carboxamide,

6-(4-fluorophenyl)-N-[2-(hydroxymethyl)-1,3-benzothiazol-5-yl]-2-methylnicotinamide,

2-bromo-N-[2-(hydroxymethyl)-1,3-benzothiazol-5-yl]-4-methyl-1,3-thiazole-5-carboxamide,

4-chloro-N-[2-(hydroxymethyl)-1,3-benzothiazol-5-yl]-2-methylbenzamide,

and salts, solvates or solvated salts thereof.

The present invention relates to the compounds of the invention as hereinbefore defined as well as to the salts, solvates or solvated salts thereof. Salts for use in pharmaceutical compositions will be pharmaceutically acceptable salts, but other salts may be useful in the production of the compounds of the invention.

A suitable pharmaceutically acceptable salt of the compounds of the invention is, for example, an acid-addition salt, for example an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.).

Some compounds of the invention may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of the invention.

Methods of Preparation

Some compounds of the present invention may be prepared according to the methods described in PCT/SE2004/000635.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). For representative examples of heterocyclic chemistry see for example “Heterocyclic Chemistry”, J. A. Joule, K. Mills, G. F. Smith, 3^{rd} ed. Chapman and Hall (1995), p. 189-224 and “Heterocyclic Chemistry”, T. L. Gilchrist, 2^{nd} ed. Longman Scientific and Technical (1992), p. 248-282.

The term “room temperature” and “ambient temperature” shall mean, unless otherwise specified, a temperature between 16 and 25° C.

One embodiment of the invention relates to processes for the preparation of the compound of formula I wherein P, R¹, R² and n, are defined as above, comprising;

a) reaction of an aromatic amine of formula (II), wherein P′ may suitably be a protecting group such as acetyl, ALLOC or BOC, with a properly substituted acyl chloride (III) optionally in the presence of a base:

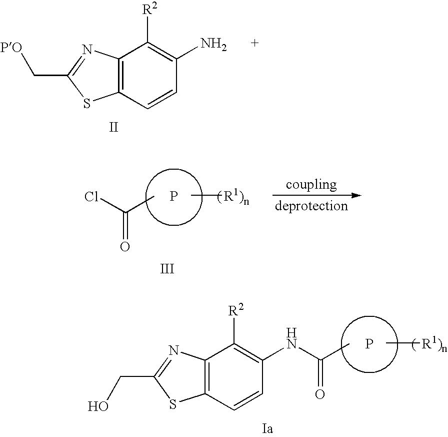

This reaction may be performed in any manner known to the skilled person in the art. Suitable solvents to be used for this reaction may be halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxan or any mixtures thereof. Catalysts such as heteroaromatic bases like pyridine and lutidine or tertiary amines like triethylamine, N-methylmorpholine and ethyl diisopropylamine or polymer bound tertiary amines like N,N-(diisopropyl)aminomethylpolystyrene resin may be used as well. The temperature may be between −40 and 40° C. and the reaction time may be between 0.5 and 30 h.

Or,

b) reaction of an aromatic amine of formula (II), wherein P′ may suitably be a protecting group such as acetyl, ALLOC or BOC, with a properly substituted acid (IV) in the presence of a coupling agent (activator) like for example 1-[3-(dimethylamino)propyl]-3-ethylcarbodiimide hydrochloride.

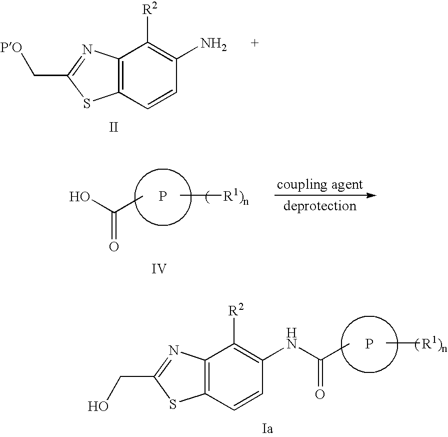

Suitable solvents to be used for this reaction may be tertiary amides such as dimethylformamide and dimethylacetamide, halogenated hydrocarbons such as chloroform, dichloromethane and dichloroethane or aromatic and heteroaromatic compounds such as benzene, toluene, xylene, pyridine and lutidine or ethers such as ethyl ether, tetrahydrofuran and dioxan or any mixtures thereof. Catalysts such as heteroaromatic bases like pyridine and lutidine or tertiary amines like triethylamine, N-methylmorpholine and ethyl diisopropylamine may be used as well. The temperature may be between 10 and 60° C. and the reaction time may be between 3 and 30 h.

Or,

c) oxidation of an intermediate Ic to the aldehydes Id

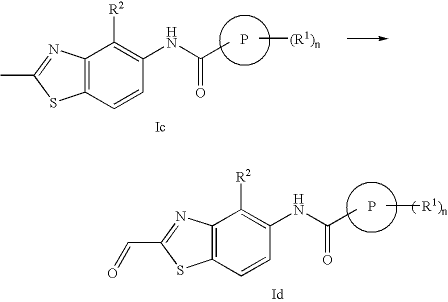

The oxidation step is accomplished by using an appropriate oxidative reagent for example, manganese dioxide, chromium trioxide or selenium dioxide. Suitable solvents to be used for this reaction may be ethers such as dioxane and tetrahydrfurane, ketones such as acetone and butan-2-one, or halogenated hydrocarbons such as chloroform, dichloromethane and dichloroethane or any mixtures thereof. The temperature may be between 0 and 80° C. and the reaction time may be between 3 and 50 h. R²=Aryl or protecting groups.

Or,

d) reduction of the aldehyde Ie to a corresponding primary alcohol

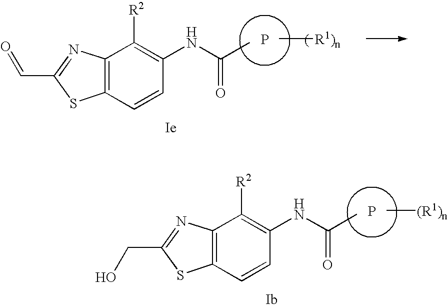

As a suitable reductive agent such as sodium borohydride may be used in a solvent like methanol or another alcohol or mixture thereof with water in a temperature range between −10 and 40° C.

Or,

e) treatment of the aldehyde Ie with organometallic reagent leading to secondary alcohols

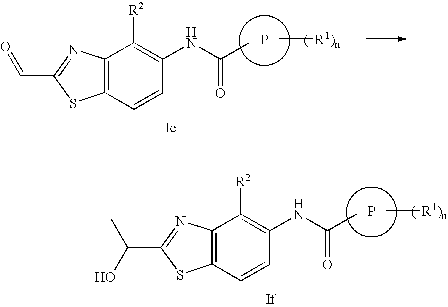

Organometalic reagent may be a magnesium derivatives like methylmagnesium bromide or organolithium compound like methyllithium and a suitable solvent may be chosen from a range of aprotic inert solvents like diethyl ether, tetrahydrofuran, benzene, etc.

Or,

f) oxidation of the 2-methyl derivative Ig and subsequent reduction of the intermediary aldehyde to the 2-hydroxymethyl derivative Ih

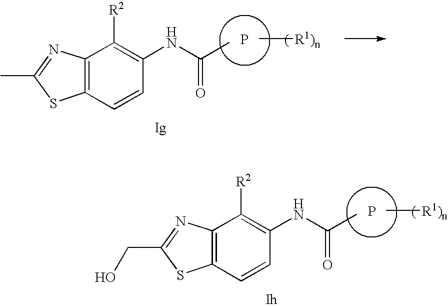

The oxidation step is accomplished by using an appropriate oxidative reagent for example, magnesium dioxide, chromium trioxide or selenium dioxide. Suitable solvents to be used for this reaction may be ketones such as acetone and butan-2-one, or halogenated hydrocarbons such as chloroform, dichloromethane and dichloroethane or any mixtures thereof. The temperature may be between 0 and 80° C. and the reaction time may be between 3 and 50 h. The subsequent reduction is typically carried out using sodium borohydride in methanol.

One embodiment of the invention relates to compounds selected from the group of

4-({[2-({[(allyloxy)carbonyl]oxy}methyl)-1,3-benzothiazol-5-yl]amino}carbonyl)-2,5-dimethylbenzoic acid,

allyl(5-amino-4-chloro-1,3-benzothiazol-2-yl)methyl carbonate,

4-tert-butoxy-2-methylbenzoic acid,

4-isopropoxy-2-methylbenzoic acid,

2-chloro-N-[2-(hydroxymethyl)-1,3-benzothiazol-5-yl]-6-(trifluoromethyl)-nicotinamide,

2-ethyl-4-isopropoxybenzoic acid,

Allyl{5-[(tert-butoxycarbonyl)amino]-1,3-benzothiazol-2-yl}methyl carbonate, and

Allyl{5-[(tert-butoxycarbonyl)amino]-4-fluoro-1,3-benzothiazol-2-yl}methyl carbonate,

Allyl(5-amino-4-fluoro-1,3-benzothiazol-2-yl)methyl carbonate,

4-bromo-2-(methoxymethyl)benzoic acid, and 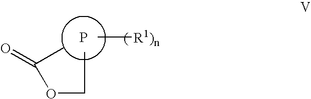
wherein P, R¹ and n are defined as in claim 1.

Another embodiment relates to the use of these compounds as intermediates in the preparation of compounds of the invention.

Pharmaceutical Composition

According to one embodiment of the present invention there is provided a pharmaceutical composition comprising as active ingredient a therapeutically effective amount of the compound of the invention, or salts, solvates or solvated salts thereof, in association with one is or more pharmaceutically acceptable diluents, excipients and/or inert carriers.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers. Suitable daily doses of the compounds of the invention in the treatment of a mammal, including man, are approximately 0.01 to 250 mg/kg bodyweight at peroral administration and about 0.001 to 250 mg/kg bodyweight at parenteral administration.

The typical daily dose of the active ingredient varies within a wide range and will depend on various factors such as the relevant indication, severity of the illness being treated, the route of administration, the age, weight and sex of the patient and the particular compound being used, and may be determined by a physician.

### Examples of Pharmaceutical Composition

The following illustrate representative pharmaceutical dosage forms containing a compound of the invention, or salts, solvates or solvated salts thereof, (hereafter compound X), for preventive or therapeutic use in mammals:

| (a): Tablet | mg/tablet |
|---|---|
| Compound X | 100 |
| Lactose | 182.75 |
| Croscarmellose sodium | 12.0 |
| Maize starch paste (5% w/v paste) | 2.25 |
| Magnesium stearate | 3.0 |

| (b): Capsule | mg/capsule |
|---|---|
| Compound X | 10 |
| Lactose | 488.5 |
| Magnesium stearate | 1.5 |

| (c): Injection | (50 mg/ml) |
|---|---|
| Compound X | 5.0% w/v |
| 1M Sodium hydroxide solution | 15.0% v/v |
| 0.1M Hydrochloric acid | (to adjust pH to 7.6) |
| Polyethylene glycol 400 | 4.5% w/v |
| Water for injection | up to 100% |

The above compositions may be obtained by conventional procedures well known in the pharmaceutical art.

Medical Use

Surprisingly, it has been found that the compounds according to the present invention are useful in therapy. The compounds of the invention, or salts, solvates or solvated salts thereof, as well as their corresponding active metabolites, exhibit a high degree of potency and selectivity for individual vanilloid receptor 1 (VR1) groups. Accordingly, the compounds of the present invention are expected to be useful in the treatment of conditions associated with excitatory activation of vanilloid receptor 1 (VR1).

The compounds may be used to produce an inhibitory effect of VR1 in mammals, including man.

VR1 are highly expressed in the peripheral nervous system and in other tissues. Thus, it is expected that the compounds of the invention are well suited for the treatment of VR1 mediated disorders.

The compounds of the invention are expected to be suitable for the treatment of acute and chronic pain, acute and chronic neuropathic pain and acute and chronic inflammatory pain.

Examples of such disorder may be selected from the group comprising low back pain, post-operative pain, visceral pains like chronic pelvic pain and the like.

Further relevant disorders may be selected from the group comprising cystitis, including interstitial cystitis and pain related thereto, ischeamic, sciatia, diabetic neuropathy, multiple sclerosis, arthritis, fibromyalgia, psoriasis, cancer, emesis, urinary incontinence, hyperactive bladder and HIV neuropathy.

Additional relevant disorders may be selected from the group comprising gastro-esophageal reflux disease (GERD), irritable bowel syndrome (IBS), inflammatory bowel disease (IBD) and pancreatitis.

Other relevant disorders are related to respiratory diseases and may be selected from the group comprising asthma, cough, chronic obstructive lung disease and emphysema, lung fibrosis and interstitial lung disease.

The VR1 inhibitor(s) may be administrated by either an oral or inhaled route. The respiratory disease may be an acute and chronic illness and may be related to infection(s) and/or exposure to environmental pollution and/or irritants.

The compounds of the invention may also be used as antitoxin to treat (over-) exposure to VR1 activators like capsaicin, tear gas, acids or heat. Regarding heat, there is a potential use for VR1 antagonists in (sun-) burn induced pain, or inflammatory pain resulting from burn injuries.

The compounds may further be used for treatment of tolerance to VR1 activators.

One embodiment of the invention relates to the compounds of the invention as hereinbefore defined, for use as a medicament.

Another embodiment of the invention relates to the compounds of the invention as hereinbefore defined, for use as a medicament for treatment of VR1 mediated disorders.

A further embodiment of the invention relates to the compounds of the invention as hereinbefore defined, for use as a medicament for treatment of acute and chronic pain disorders.

Yet another embodiment of the invention relates to the compounds of the invention as hereinbefore defined, for use as a medicament for treatment of acute and chronic neuropathic pain.

Yet a further embodiment of the invention relates to the compounds of the invention as hereinbefore defined, for use as a medicament for treatment of acute and chronic inflammatory pain.

One embodiment of the invention relates to the compounds of the invention as hereinbefore defined, for use as a medicament for treatment of low back pain, post-operative pain and visceral pains like chronic pelvic pain.

Another embodiment of the invention relates to the compounds of the invention as hereinbefore defined, for use as a medicament for treatment of cystitis, including interstitial cystitis and pain related thereto, ischeamic, sciatia, diabetic neuropathy, multiple sclerosis, arthritis, fibromyalgia, psoriasis, cancer, emesis, urinary incontinence, hyperactive bladder and HIV neuropathy.

A further embodiment of the invention relates to the compounds of the invention as hereinbefore defined, for use as a medicament for treatment of gastro-esophageal reflux disease (GERD), irritable bowel syndrome (IBS), inflammatory bowel disease (IBD) and pancreatitis.

Yet a further embodiment of the invention relates to the compounds of the invention as hereinbefore defined, for use as a medicament for treatment of respiratory diseases selected from the group comprising asthma, cough, chronic obstructive lung disease and emphysema, lung fibrosis and interstitial lung disease.

One embodiment of the invention relates to the use of the compound of the invention as hereinbefore defined, in the manufacture of a medicament for treatment of VR1 mediated disorders and for treatment of acute and chronic pain disorders, acute and chronic neuropathic pain and acute and chronic inflammatory pain, and respiratory diseases and any other disorder mentioned above.

Another embodiment of the invention relates to a method of treatment of VR1 mediated disorders and acute and chronic pain disorders, acute and chronic neuropathic pain and acute and chronic inflammatory pain, and respiratory diseases, and any other disorder mentioned above, comprising administering to a mammal, including man in need of such treatment, a therapeutically effective amount of the compounds of the invention, as hereinbefore defined.

A further embodiment of the invention relates to a pharmaceutical composition comprising a compound of the invention as hereinbefore defined, for use in treatment of VR1 mediated disorders and for treatment of acute and chronic pain disorders, acute and chronic neuropathic pain and acute and chronic inflammatory pain, and respiratory diseases, and any other disorder mentioned above.

In the context of the present specification, the term “therapy” and “treatment” includes prevention and prophylaxis, unless there are specific indications to the contrary. The terms “treat”, “therapeutic” and “therapeutically” should be construed accordingly.

In this specification, unless stated otherwise, the term “inhibitor” and “antagonist” mean a compound that by any means, partly or completely, blocks the transduction pathway leading to the production of a response by the ligand.

The term “disorder”, unless stated otherwise, means any condition and disease associated with vanilloid receptor activity.

Non-Medical Use

In addition to their use in therapeutic medicine, the compounds of the invention, or salts, solvates or solvated salts thereof, are also useful as pharmacological tools in the development and standardisation of in vitro and in vivo test systems for the evaluation of the effects of inhibitors of VR1 related activity in laboratory animals such as cats, dogs, rabbits, monkeys, rats and mice, as part of the search for new therapeutics agents.

### EXAMPLES

The invention will now be illustrated by the following non-limiting examples.

General Methods

The invention will now be illustrated by the following Examples in which, generally:

(i) operations were carried out at ambient or room temperature, i.e. in the range 17 to 25° C. and under an atmosphere of an inert gas such as argon unless otherwise stated;

(ii) evaporations were carried out by rotary evaporation in vacuo and work-up procedures were carried out after removal of residual solids by filtration;

(iii) column chromatography (by the flash procedure) was performed on Silicycle silica gel (grade 230-400 mesh, 60 Å, cat. Numb. R100301B) or obtained from Silicycle, Quebec, Canada or high pressure liquid chromatography (HPLC) was performed on C18 reverse phase silica, for example on a Phenomenex, Luna C-18 100 Å preparative reversed-phase column;

(iv) The ¹H NMR spectra were recorded on Brucker at 400 MHz. The mass spectra were recorded utilising electrospray (LC-MS; LC:Waters 2790, column XTerra MS C₈ 2.5 μm 2.1×30 mm, buffer gradient H₂O+0.1% TFA:CH₃CN+0.04% TFA, MS:micromass ZMD//ammonium acetate buffer) ionisation techniques;

(v) yields, where present, are not necessarily the maximum attainable;

(vi) intermediates were not necessarily fully purified but their structures and purity were assessed by thin layer chromatographic, HPLC and/or NMR analysis

(vii) the following abbreviations have been used:
ALLOC allyloxycarbonyl
DCE dichloroethane
DCM dichloromethane
DMAP dimethylaminopyridine
EDC 1-(3-dimethylaminopropyl)-3-ethylcarbodiimide hydrochloride
HATU O-(7-azabenzotriazol-1-yl)-N,N,N′,N′-tetramethyluronium hexafluorophosphate
HPLC high performance liquid chromatography
LC liquid chromatography
MsCl methanesulfonyl chloride
MS mass spectometry
ret. time retention time
TEA triethyl amine
TFA trifluroacetic acid
THF tetrahydrofurane
DMF dimethylformamide
TMEDA tetramethylethylenediamine
EtOAc ethyl acetate
BuLi Butyl lithium
TMEDA tetramethylethylenediamine

### Intermediate 1

### 4-({[2-(hydroxymethyl)-1,3-benzothiazol-5-yl]amino}carbonyl)benzoic acid

According to amide bond-forming procedure a), the amine (1.00 g, 3.78 mmol), monomethyl terephtalate (681 mg, 3.78 mmol), EDC (1.451 g, 7.57 mmol), and DMAP (925 mg, 7.57 mmol) were mixed in DCM (50.0 mL) and DMF (20.0 mL). The mixture was worked up as usual to yield the amide. The product was purified by flash chromatography eluting with mixtures of hexanes and ethyl acetate (2:1, 100% ethyl acetate) to yield the methyl ester. The suspension of methyl ester in 1M NaOH (35 mL) is then heated to 95° C. for 25 minutes. After cooling the reaction is acidified with 10% HCl and the precipitate is filtrated. The product was purified by Gilson HPLC (Luna 15 u, C18 (2), 250 mm×21.2 mm) eluting with mixtures of MeCN and H₂O containing 0.1% TFA to yield the title compound (1.067 g, 3.25 mmol, 86%).

### Intermediate 2

### Allyl(5-amino-4-chloro-1,3-benzothiazol-2-yl)methyl carbonate

Allyl(5-amino-1,3-benzothiazol-2-yl)methyl carbonate (500 mg, 1.89 mmol) was dissolved in DCM (19.0 mL) and N-chlorosuccinimide (253 mg, 1.89 mmol) was added. The mixture was stirred until the reaction appeared complete by LC-MS. The solution was concentrated under reduced pressure and purified by flash chromatography eluting with mixtures of hexanes and EtOAc (4:1, 2:1) to yield the title compound (429 mg, 1.44 mmol, 76%). ¹H NMR (400 MHz, CHLOROFORM-D) δ ppm 2.77 (s, 2H) 4.71 (dt, J=5.86, 2.73, 1.37 Hz, 2 H) 5.27-5.46 (m, 2 H) 5.57 (s, 2 H) 5.89-6.05 (m, 1 H) 6.92 (d, J=8.59 Hz, 1 H) 7.55 (d, J=8.59 Hz, 1 H).

### Intermediate 3

### 4-tert-butoxy-2-methylbenzoic acid

A solution of 4-tert-butoxybenzoic acid (500 mg, 2.57 mmol) in THF (10.0 mL) was cooled to −78° C. A solution of sBuLi (7.84 mmol, 5.60 mL, 1.4 M in cyclohexane) and TMEDA (930 mg, 8.00 mmol, 1.20 mL) in THF (10.0 mL) was cooled to −78° C. and added drop-wise over 20 minutes to the first solution. The mixture was stirred at −78° C. for 1 hour, and then iodomethane (2.27 g, 16.0 mmol, 1.00 mL) was added. The mixture was slowly warmed to room temperature and stirred for 16 hours. The reaction was quenched with 0.5 N HCl, and the aqueous phase was extracted with EtOAc. The combined organic phases were dried with Na₂SO₄, filtered and concentrated. The product was purified by flash chromatography on silica gel eluting with a mixture of hexanes and EtOAc (9:1), (107 mg, 0.510 mmol, 20%). ¹H NMR (400 MHz, CHLOROFORM-D) δ ppm 1.42 (s, 9 H) 2.63 (s, 3 H) 6.85-6.90 (m, 2 H) 8.02 (d, J=8.40 Hz, 1 H).

### Intermediate 4

### 4-isopropoxy-2-methylbenzoic acid

According to the procedure outlined for example 6, 4-isopropoxybenzoic acid (1.00 g, 5.54 mmol), tBuLi (12.2 mmol, 7.20 mL, 1.70 M in pentane), TMEDA (1.40 g, 12.2 mmol, 1.84 mL) and iodomethane (3.14 g, 22.2 mmol, 1.40 mL) were mixed in THF (10.0 mL). The product was purified by flash chromatography on silica gel eluting with a mixture of hexanes and EtOAc (9:1) (144 mg, 0.740 mmol, 13.4%). ¹H NMR (400 MHz, CHLOROFORM-D) δ ppm 1.36 (d, J=6.05 Hz, 6 H) 2.63 (s, 3 H) 4.64 (dt, J=12.16, 6.13 Hz, 1 H) 6.73-6.77 (m, 2 H) 8.06 (d, J=9.18 Hz, 1 H).

### Intermediate 5

### 2-chloro-N-[2-(hydroxymethyl)-1,3-benzothiazol-5-yl]-6-(trifluoromethyl)-nicotinamide

A mixture of 5-amino-1,3-benzothiazol-2-yl)methanol (518 mg, 2.0 mmol), 2-chloro-6-(trifluoromethyl)-nicotinic acid (529 mg, 2.35 mmol), EDC (450 mg, 2.35 mmol), DMAP (288 mg, 2.35 mmol) and TEA (328 μL, 2.35 mmol) in DCM (10.0 mL) was stirred at room temperature for 16 hours. The reaction was quenched with water (10.0 mL) and the phases were partitioned. The organic phase was washed with brine solution (10.0 mL), dried with sodium sulfate and concentrated to a residue by rotary evaporator. The material was used in the following step without further purification.

### Mixture of Intermediate 4 and Intermediate 6, Where Intermediate 6=2-ethyl-4-isopropoxybenzoic acid

### 2-ethyl-4-isopropoxybenzoic acid

A 500 mL flask with a magnetic stirring bar was dried in the oven, flushed with N₂ and charged with 9.8 mL of TMEDA (distilled over CaH₂) in 45 mL of dry THF. This solution was cooled to −95° C. (acetone/liquid nitrogen bath) and stirred for 10 min. Sec-butyllithium (1.1 M in cyclohexane, 59.6 mL, 65.6 mmol, 2.2 equiv) was added and the mixture stirred for 20 min. 4-Isopropoxybenzoic acid (5.377 g, 29.8 mmol, 1 equiv) dissolved in 30 mL of dry THF was added dropwise over 30 min. The temperature of the bath was raised to −78° C. (dry ice/acetone), and the mixture stirred for 45 min. Iodomethane (7.42 mL, 119.2 mmol, 4 equiv) was added. The temperature was raised slowly to room temperature and the reaction was quenched with water (100 mL). The phases were separated and the organic phase extracted with NaOH 2M. The reunited organic phases were washed with diethylether and acidified with concentrated HCl (formation of a precipitate). Diethylether was added, the phases were separated and the aqueous phase extracted with 3 portions of diethylether. The reunited organic phases were dried over magnesium sulfate, filtered and evaporated to dryness. The crude product was then purified by column chromatography (SiO₂, CH₂Cl₂/AcOEt 4:1), giving a white solid (3.821 g, 66%). This contains 2 compounds: 4-isopropoxy-2-methylbenzoic acid (main product; intermediate 4) and 2-ethyl-4-isopropoxybenzoic acid (by-product, ˜10%).

### 2-ethyl-4-isopropoxybenzoic acid intermediate 6

¹H NMR (400 MHz, CD3OD, δ ppm): 1.19 (t, J=7.52 Hz, 3 H); 1.32 (d, J=6.05 Hz, 6 H); 2.98 (q, J=7.42 Hz, 2 H); 4.67 (septet, J=6.10 Hz, 1 H); 6.72-6.79 (m, 2 H); 7.88 (d, J=9.37 Hz, 1 H).

### Intermediate 7

### Allyl{5-[(tert-butoxycarbonyl)amino]-1,3-benzothiazol-2-yl}methyl carbonate

Allyl(5-amino-1,3-benzothiazol-2-yl)methyl carbonate (5.00 g, 18.92 mmol) and di-tert-butylcarbonate (6.19 g, 28.38 mmol) were combined and heated at 70° C. for 45 minutes with stirring. The reaction mixture became a homogeneous solution with gas evolution. The solution was cooled to room temperature, diluted with ethyl acetate and washed with saturated aqueous sodium bicarbonate. The organic layer was dried (MgSO₄), filtered and concentrated. Purification by flash chromatography (0% to 30% ethyl acetate in heptane) afforded a 3:1 mixture of Allyl(5-amino-4-fluoro-1,3-benzothiazol-2-yl)methyl carbonate and the di-Boc product as a yellow oil (7.11 g, 77% yield of Allyl(5-amino-4-fluoro-1,3-benzothiazol-2-yl)methyl carbonate). The material was used without further purification in the next step. Purity (HPLC-254 nm): >75%. M.S. (calcd): 365.1 (MH⁺), M.S (found): 364.9 (MH⁺).

### Intermediate 8

### Allyl{5-[(tert-butoxycarbonyl)amino]-4-fluoro-1,3-benzothiazol-2-yl}methyl carbonate

To a solution of Allyl(5-amino-4-fluoro-1,3-benzothiazol-2-yl)methyl carbonate (7.00 g, 14.4 mmol) in acetonitrile (100 mL) was added Selectfluor (10 g, 28.23 mmol). The reaction was stirred overnight at room temperature and then concentrated in vacuo. The residue was dissolved in ethyl acetate and washed with saturated aqueous sodium bicarbonate and water, then dried (MgSO₄), filtered and concentrated. The residue was purified by flash chromatography eluting with 0% to 20% ethyl acetate in heptane to give the title compound as a yellow oil (1.96 g, 36%). ¹H NMR (400 MHz, CDCl₃) δ ppm 1.55 (s, 9H), 4.69-4.75 (m, 2H), 5.29-5.35 (m, 1H), 5.37-5.46 (m, 1H), 5.56 (s, 2H), 5.90-6.03 (m, 1 H), 6.82-6.89 (m, 1H), 7.61 (dd, J=8.98, 1.37 Hz, 1H), 8.20-8.30 (m, 1H). M.S. (calcd): 383.1 (MH⁺), M.S (found): 382.9 (MH⁺).

### Intermediate 9

### Allyl(5-amino-4-fluoro-1,3-benzothiazol-2-yl)methyl carbonate

Allyl{5-[(tert-butoxycarbonyl)amino]-4-fluoro-1,3-benzothiazol-2-yl}methyl carbonate (1.96 g, 5.13 mmol) was dissolved in dichloromethane (50 mL) and trifluoroacetic acid (4 mL) was added. The reaction was stirred overnight and then concentrated in vacuo to afford the TFA salt of the title compound as a yellow oil (2.00 g, 98%). ¹H NMR (400 MHz, CDCl₃) δ ppm 4.70-4.73 (m, 2H), 5.30-5.35 (m, 1H), 5.37-5.45 (m, 1H), 5.59-5.61 (m, 2H), 5.90-6.02 (m, 1H), 7.02 (dd, J=8.59, 7.62 Hz, 1H), 7.46 (dd, J=8.59, 1.17 Hz, 1H). M.S. (calcd): 283.0 (MH⁺), M.S (found): 282.9 (MH⁺).

### Intermediate 10

### 4-bromo-2-(methoxymethyl)benzoic acid

The nitrile, 4-bromo-2-methylbenzonitrile (750 mg, 3.83 mmol), N-bromosuccinimide (2.0 g, 26.0 mmol) and 1,1′azobis(cyclohexanecarbonitrile) (100 mg, 0.410 mmol) dissolved in 30.0 ml of carbon tetrachloride was refluxed 24 hours, the resulting reaction mixture filtered and concentrated. The resulting residue was then dissolved in a 25% sodium methoxide solution in methanol (10 mL) stirred 2 hours at room temperature. This mixture was then concentrated, partitioned between water and ethyl acetate. The organic layer was separated then concentrated under reduced pressure. The resulting residue was purify on silicagel (heptane/ethyl acetate 9:1) to yield (320 mg) of the intermediate 4-bromo-2-(methoxymethyl)benzonitrile. This residue was dissolved in isobutanol (4.0 mL) heated to reflux. Distilled water (0.5 mL) and sodium hydroxide (400 mg, 10 mmol) were added. The reaction mixture was stirred at this temperature for 48 hours. The solvents were then evaporated, the resulting residue dissolved in distilled water (5.0 mL). This solution was acidified with aqueous 1N HCl, a pale red solid formed which was filtered and dried (200 mg, 27%). Trituration with ethyl acetate gave a pale solid (34 mg, 4%). 1H NMR (600 MHz, DMSO-D6) δ ppm 3.38 (s, 3 H) 4.75 (s, 2 H) 7.60 (dd, J=8.32, 1.66 Hz, 1 H) 7.74 (s, 1 H) 7.80 (d, J=8.45 Hz, 1 H).

### Example 1

### 3-tert-butoxy-N-[2-(hydroxymethyl)-1,3-benzothiazol-5-yl]benzamide

The amine, allyl{5-[(tert-butoxycarbonyl)amino]-1,3-benzothiazol-2-yl}methyl carbonate (200 mg, 0.757 mmol), 3-tert-butoxybenzoic acid (147 mg, 0.757 mmol), EDC (290 mg, 1.51 mmol), and DMAP (185 mg, 1.51 mmol) were mixed in DCM (10.0 mL) and DMF (10.0 mL). The mixture was stirred for 18 hours, and the solvents were evaporated. The residue was dissolved in DCM and washed with a saturated solution of NaHCO₃. The mixture was dried with Na₂SO₄, filtered and concentrated under reduced pressure. The crude amide product was mixed with aqueous 1M NaOH (10.0 mL) and THF (10.00 mL) for removal of the alloc protecting group. The aqueous phase was extracted with DCM. The organic phases were collected, dried with Na₂SO₄, filtered and concentrated under reduced pressure. The product was purified by flash chromatography eluting with mixtures of hexanes and ethyl acetate (2:1, 1:1) to yield the title compound (179 mg, 0.502 mmol, 66%). ¹H NMR (400 MHz, METHANOL-D4) δ ppm 1.39 (s, 9 H) 4.95 (s, 2 H) 7.23 (dd, J=8.01, 1.56 Hz, 1 H) 7.43 (t, J=7.91 Hz, 1 H) 7.58 (s, 1 H) 7.69 (d, J=7.81 Hz, 1 H) 7.72 (dd, J=8.79, 1.95 Hz, 1 H) 7.95 (d, J=8.79 Hz, 1 H) 8.40 (d, J=1.56 Hz, 1 H). MS [MH+] calc. 357.1, found 357.0. Anal found C 64.50%, H 5.76%, N 7.79%.

### Example 2

### 4-(dimethylamino)-N-[2-(hydroxymethyl)-1,3-benzothiazol-5-yl]benzamide

A mixture of allyl(5-amino-1,3-benzothiazol-2-yl)methyl carbonate (0.946 mmol, 250 mg), 4-(dimethylamino)benzoyl chloride (0.946 mmol, 174 mg), triethylamine (0.946 mmol, 132 uL), and 4-dimethylamino-pyridine (0.946 mmol, 116 mg), was stirred in dichloromethane (15.0 mL) at room temperature for 16 hours. The reaction was quenched with water (20.0 mL) and extracted with dichloromethane (2×10.0 mL). The organic phases were combined and washed with brine solution (15.0 mL). The organic was dried with anhydrous sodium sulphate, and filtered to remove the solids. The filtrate was concentrated by rotary evaporator to yield a residue that was dissolved in methanol (10.0 mL), and treated with aqueous sodium hydroxide (1.0 M, 1.0 mL). The mixture was stirred for 1 hour, then the solvents was removed by rotary evaporator to give a residue that was purified by Gilson HPLC (Luna 15 um, C18 (2), 250 mm×21.2 mm) eluting with mixtures of MeCN and H₂O containing 0.1% TFA to give the title compound. ¹H NMR (400 MHz, METHANOL-D4) δ ppm 3.06 (s, 6 H) 4.94 (s, 2 H) 6.84 (d, J=8.79 Hz, 2 H) 7.70 (dd, J=8.59, 1.95 Hz, 1 H) 7.88 (d, J=8.98 Hz, 2 H) 7.92 (d, J=8.59 Hz, 1 H) 8.37 (d, J=1.95 Hz, 1 H). (M+1)=328.0, (M+1) calc. =328.1.

Compounds in the following examples were synthesized according to the amide bond-forming procedures described in the example 1 or 2 starting from an appropriate and aromatic amine (corresponding to intermediate 9) or synthesized according to the procedures described in PCT/SE2004/000635, and an appropriately substituted commercially available aromatic acid or an aromatic acyl chloride. The amide bond-forming procedures were followed by the deprotection as described in Example 2.

| Example | | MS | MS | |
|---|---|---|---|---|
| Nr | Name | Calc. | found | 1H NMR |
| 3 | tert-butyl 4-({[2-(hydroxymethyl)- | 385.1 | 385.0 | (400 MHz, |
| | 1,3-benzothiazol-5- | | | METHANOL-D4) δ |
| | yl]amino}carbonyl)benzoate. | | | ppm 1.62 (s, 9H) |
| | | | | 4.95 (s, 2H) 7.74 (dd, J = 8.69, |
| | | | | 1.86 Hz, 1H) 7.97 (d, |
| | | | | J = 8.59 Hz, 1H) |
| | | | | 8.00-8.04 (m, 2H) |
| | | | | 8.06-8.11 (m, 2H) 8.43 (d, J = 1.56 Hz, |
| | | | | 1H) |
| 4 | N,N-diethyl-N′-[2- | 384.1 | 384.0 | (400 MHz, |
| | (hydroxymethyl)-1,3-benzothiazol- | | | METHANOL-D4) δ |
| | 5-yl]terephthalamide | | | ppm 1.14 (t, J = 6.93 Hz, |
| | | | | 3H) 1.21-1.31 (m, 3H) |
| | | | | 3.26-3.34 (m, 2H) |
| | | | | 3.58 (q, J = 6.83 Hz, 2H) |
| | | | | 4.96 (s, 2H) 7.53 (d, J = 8.20 Hz, |
| | | | | 2H) 7.74 (dd, |
| | | | | J = 8.69, 1.85 Hz, 1H) |
| | | | | 7.97 (d, J = 8.79 Hz, 1H) |
| | | | | 8.04 (d, J = 8.20 Hz, 2H) |
| | | | | 8.43 (d, J = 1.76 Hz, 1H). |
| 5 | N-[2-(hydroxymethyl)-1,3- | 401.1 | 400.8 | ¹H NMR (400 MHz, |
| | benzothiazol-5-yl]-4-(1,1,2,2- | | | METHANOL-D4) δ |
| | tetrafluoroethoxy)benzamide. | | | ppm 4.95 (s, 2H) |
| | | | | 6.37 (m, 1H) 7.40 (d, J = 8.59 Hz, |
| | | | | 2H) 7.69-7.76 (m, |
| | | | | 1H) 7.96 (d, J = 8.59 Hz, |
| | | | | 1H) 8.05 (d, J = 8.79 Hz, |
| | | | | 2H) 8.38-8.43 (m, 1H). |
| 6 | 4-Cyclohexyl-N-[2- | 367.1 | 367.0 | (400 MHz, |
| | (hydroxymethyl)-1,3-benzothiazol- | | | METHANOL-D4) δ |
| | 5-yl]benzamide | | | ppm 1.25-1.38 (m, 1H), |
| | | | | 1.38-1.56 (m, 4H), |
| | | | | 1.73-1.81 (m, 1H), |
| | | | | 1.81-1.93 (m, 4H), |
| | | | | 2.55-2.66 (m, 1H), |
| | | | | 4.95 (s, 2H), 7.37 (d, |
| | | | | J = 8.40 Hz, 2H), |
| | | | | 7.72 (dd, J = 8.59, 1.95 Hz, 1H), |
| | | | | 7.88 (d, J = 8.20 Hz, 2H), |
| | | | | 7.94 (d, J = 8.79 Hz, 1H), |
| | | | | 8.40 (d, J = 1.95 Hz, 1H). |
| 7 | 3-Fluoro-N-[2-(hydroxymethyl)- | 371.0 | 371.0 | (400 MHz, |
| | 1,3-benzothiazol-5-yl]-4- | | | METHANOL-D4) δ |
| | (trifluoromethyl)benzamide | | | ppm 4.95 (s, 2H) |
| | | | | 7.73 (dd, J = 8.69, 1.86 Hz, 1H) |
| | | | | 7.83-8.01 (m, 4H) |
| | | | | 8.43 (d, J = 1.76 Hz, 1H) |
| 8 | N-[2-(hydroxymethyl)-1,3- | 354.1 | 353.8 | (400 MHz, DMSO-D6) |
| | benzothiazol-5-yl]-6- | | | δ ppm 4.84 (s, 2H), |
| | (trifluoromethyl)nicotinamide | | | 6.26 (s, 1H), 7.70-7.78 (m, 1H), |
| | | | | 8.02-8.07 (m, H), |
| | | | | 8.08-8.12 (m, 1H), |
| | | | | 8.43 (d, J = 1.76 Hz, 1H), |
| | | | | 8.59 (dd, J = 8.10, 1.66 Hz, |
| | | | | 1H), 9.27 (d, J = 0.98 Hz, |
| | | | | 1H), 10.83 (s, 1H) |
| 9 | N-[2-(hydroxymethyl)-1,3- | 369.1 | 369.0 | (400 MHz, DMSO-D6) |
| | benzothiazol-5-yl]-6-piperidin-1- | | | δ ppm 1.47-1.70 (m, 6H) |
| | ylnicotinamide | | | 3.58-3.72 (m, 4H) |
| | | | | 4.83 (s, 2H) 6.98 (d, |
| | | | | J = 8.98 Hz, 1H) |
| | | | | 7.72 (dd, J = 8.79, 1.95 Hz, 1H) |
| | | | | 7.99 (d, J = 8.79 Hz, 1H) |
| | | | | 8.11 (dd, J = 9.18, 2.15 Hz, |
| | | | | 1H) 8.40 (d, J = 1.76 Hz, |
| | | | | 1H) 8.69 (d, J = 2.34 Hz, |
| | | | | 1H) 10.18 (s, 1H) |
| 10 | 4-(dimethylamino)-N-[2- | 378.1 | 378.0 | (400 MHz, |
| | (hydroxymethyl)-1,3-benzothiazol- | | | METHANOL-D4) δ |
| | 5-yl]-1-naphthamide | | | ppm 3.10 (s, 6H), |
| | | | | 4.96 (s, 2H), 7.42 (d, J = 7.81 Hz, |
| | | | | 1H), 7.59-7.66 (m, |
| | | | | 2H), 7.72 (d, J = 7.62 Hz, |
| | | | | 1H), 7.78 (d, J = 7.81 Hz, |
| | | | | 1H), 7.97 (d, J = 8.79 Hz, |
| | | | | 1H), 8.23-8.28 (m, 1H), |
| | | | | 8.30-8.36 (m, 1H), |
| | | | | 8.49 (s, 1H) |
| 11 | 2-fluoro-N-[2-(hydroxymethyl)- | 371.0 | 371.0 | (400 MHz, DMSO-D6) |
| | 1,3-benzothiazol-5-yl]-4- | | | δ ppm 4.81-4.87 (m, 2H), |
| | (trifluoromethyl)benzamide | | | 6.18-6.31 (m, 1H), |
| | | | | 7.65 (dd, J = 8.69, 1.85 Hz, |
| | | | | 1H), 7.73 (d, J = 8.01 Hz, |
| | | | | 1H), 7.85-7.96 (m, |
| | | | | 2H), 8.04 (d, J = 8.59 Hz, |
| | | | | 1H), 8.38 (d, J = 1.76 Hz, |
| | | | | 1H), 10.80 (s, 1H) |
| 12 | N-[2-(hydroxymethyl)-1,3- | 384.1 | 384.0 | (400 MHz, DMSO-D6) |
| | benzothiazol-5-yl]-2-methoxy-6- | | | δ ppm 4.00 (s, 3H), |
| | (trifluoromethyl)nicotinamide | | | 7.59-7.69 (m, 2H), 8.02 (d, |
| | | | | J = 8.59 Hz, 1H), 8.25 (d, |
| | | | | J = 7.42 Hz, 1H), 8.39 (d, |
| | | | | J = 1.37 Hz, 1H), |
| | | | | 10.57 (s, 1H) |
| 13 | N-[2-(hydroxymethyl)-1,3- | 368.1 | 367.7 | (400 MHz, DMSO-D6) |
| | benzothiazol-5-yl]-2-methyl-6- | | | δ ppm 2.27 (s, 3H), |
| | (trifluoromethyl)nicotinamide | | | 4.48 (s, 2H), 7.29 (dd, |
| | | | | J = 8.79, 1.95 Hz, 1H), |
| | | | | 7.51 (d, J = 8.01 Hz, 1H), |
| | | | | 7.67 (d, J = 8.59 Hz, 1H), |
| | | | | 7.84 (d, J = 7.81 Hz, 1H), |
| | | | | 8.02 (d, J = 1.76 Hz, 1H), |
| | | | | 10.42 (s, 1H) |
| 14 | 3-fluoro-N-[2-(hydroxymethyl)- | 331.1 | 331.0 | (400 MHz, |
| | 1,3-benzothiazol-5-yl]-2,4- | | | METHANOL-D4) δ |
| | dimethylbenzamide | | | ppm 2.30 (d, J = 1.76 Hz, |
| | | | | 3H) 2.35 (d, J = 2.15 Hz, |
| | | | | 3H) 4.95 (s, 2H) |
| | | | | 7.15-7.25 (m, 2H) 7.66 (dd, |
| | | | | J = 8.59, 1.76 Hz, 1H) |
| | | | | 7.95 (d, J = 8.79 Hz, 1H) |
| | | | | 8.40 (d, J = 1.56 Hz, 1H) |
| 15 | N-[2-(hydroxymethyl)-1,3- | 313.1 | 313.0 | (400 MHz, |
| | benzothiazol-5-yl]-2,4- | | | METHANOL-D4) δ |
| | dimethylbenzamide | | | ppm 2.35 (s, 3H) |
| | | | | 2.44 (s, 3H) 4.95 (s, 2H) |
| | | | | 7.08-7.15 (m, 2H) |
| | | | | 7.39 (d, J = 7.62 Hz, 1H) |
| | | | | 7.66 (dd, J = 8.59, 1.37 Hz, 1H) |
| | | | | 7.93 (d, J = 8.59 Hz, 1H) |
| | | | | 8.40 (d, J = 1.17 Hz, 1H) |
| 16 | 4-tert-butoxy-N-[4-chloro-2- | 391.1 | 391.0 | (400 MHz, |
| | (hydroxymethyl)-1,3-benzothiazol- | | | METHANOL-D4) δ |
| | 5-yl]benzamide | | | ppm 1.42 (s, 9H) |
| | | | | 4.98 (s, 2H) 7.13 (d, J = 8.59 Hz, |
| | | | | 2H) 7.75 (d, J = 8.59 Hz, |
| | | | | 1H) 7.88-7.98 (m, |
| | | | | 3H) |
| 17 | 4-(tert-Butoxymethyl)-N-[4-chloro- | 405.1 | 405.0 | (400 MHz, |
| | 2-(hydroxymethyl)-1,3- | | | METHANOL-D4) δ |
| | benzothiazol-5-yl]benzamide | | | ppm 1.26-1.36 (m, 9H) |
| | | | | 4.57 (s, 2H) 4.98 (s, 2H) |
| | | | | 5.48 (s, 1H) 7.51 (d, |
| | | | | J = 8.20 Hz, 2H) 7.75 (d, |
| | | | | J = 8.59 Hz, 1H) |
| | | | | 7.97 (dd, J = 8.40, 2.73 Hz, 3H) |
| 18 | N-[4-chloro-2-(hydroxymethyl)- | 377.1 | 377.0 | 1.36 (d, J = 6.05 Hz, 6H) |
| | 1,3-benzothiazol-5-yl]-4- | | | 4.69-4.77 (m, 1H) |
| | isopropoxybenzamide | | | 4.99 (s, 2H) 7.03 (d, J = 8.79 Hz, |
| | | | | 2H) 7.76 (d, J = 8.59 Hz, |
| | | | | 1H) 7.97 (dd, |
| | | | | J = 8.79, 2.34 Hz, 3H) |
| 19 | 3-tert-butoxy-N-[4-chloro-2- | 391.1 | 391.0 | (400 MHz, |
| | (hydroxymethyl)-1,3-benzothiazol- | | | METHANOL-D4) δ |
| | 5-yl]benzamide. | | | ppm 1.39 (s, 9H) |
| | | | | 4.99 (s, 2H) 7.26 (dd, J = 8.01, |
| | | | | 1.76 Hz, 1H) 7.46 (t, |
| | | | | J = 7.91 Hz, 1H) 7.63 (s, |
| | | | | 1H) 7.75 (dd, J = 8.40, |
| | | | | 5.08 Hz, 2H) 7.98 (d, |
| | | | | J = 8.59 Hz, 1H) |
| 20 | tert-butyl 4-({[4-chloro-2- | 419.1 | 418.7 | (400 MHz, |
| | (hydroxymethyl)-1,3-benzothiazol- | | | METHANOL-D4) δ |
| | 5-yl]amino}carbonyl)benzoate | | | ppm 1.62 (s, 9H) |
| | | | | 4.99 (s, 2H) 7.74 (d, J = 8.59 Hz, |
| | | | | 1H) 7.99 (d, J = 8.59 Hz, |
| | | | | 1H) 8.06-8.13 (m, |
| | | | | 4H) |
| 21 | 4-Bromo-2-chloro-N-[2- | 397.0 | 396.7 | (400 MHz, DMSO-D6) |
| | (hydroxymethyl)-1,3-benzothiazol- | | | δ ppm 4.82 (s, 2H) |
| | 5-yl]benzamide. | | | 6.23 (t, J = 12.11, 6.05 Hz, 1H) |
| | | | | 7.58 (d, J = 8.20 Hz, 1H) |
| | | | | 7.63 (dt, J = 8.79, |
| | | | | 3.32, 2.15 Hz, 1H) |
| | | | | 7.68 (dd, J = 8.20, 1.95 Hz, 1H) |
| | | | | 7.88 (d, J = 1.95 Hz, 1H) |
| | | | | 8.01 (d, J = 8.59 Hz, 1H) |
| | | | | 8.35 (t, J = 1.66 Hz, 1H) |
| | | | | 10.70 (s, 1H) |
| 22 | 4-Bromo-2-fluoro-N-[2- | 381.0 | 381.0 | (400 MHz, |
| | (hydroxymethyl)-1,3-benzothiazol- | | | CHLOROFORM-D) δ |
| | 5-yl]benzamide | | | ppm 5.09 (s, 2H) |
| | | | | 7.43 (d, J = 11.52 Hz, 1H) |
| | | | | 7.50 (dd, J = 8.40, 1.56 Hz, |
| | | | | 1H) 7.71 (s, 1H) |
| | | | | 7.88 (d, J = 8.59 Hz, 1H) |
| | | | | 8.11 (t, J = 8.49 Hz, 1H) |
| | | | | 8.33 (s, 1H) 8.52 (s, 1H) |
| 23 | 4-tert-butoxy-N-[2- | 371.1 | 371.0 | (400 MHz, |
| | (hydroxymethyl)-1,3-benzothiazol- | | | CHLOROFORM-D) δ |
| | 5-yl]-2-methylbenzamide | | | ppm 1.35 (s, 9H) |
| | | | | 2.42 (s, 3H) 4.92 (s, 2H) |
| | | | | 6.77-6.86 (m, 2H) |
| | | | | 7.39 (d, J = 8.20 Hz, 1H) |
| | | | | 7.64-7.76 (m, 2H) 8.01 (s, 1H) |
| | | | | 8.20 (s, 1H) |
| 24 | N-[2-(hydroxymethyl)-1,3- | 357.1 | 357.0 | (400 MHz, DMSO-D6) |
| | benzothiazol-5-yl]-4-isopropoxy-2- | | | δ ppm 1.66 (s, 3H) |
| | methylbenzamide | | | 3.02 (s, 3H) 4.14 (s, 2H) |
| | | | | 5.92-6.15 (m, 2H) |
| | | | | 6.67 (d, J = 8.01 Hz, 1H) |
| | | | | 6.85 (d, J = 8.79 Hz, 1H) |
| | | | | 7.12 (d, J = 8.79 Hz, 1H) |
| | | | | 7.58 (s, 1H) |
| 25 | N-[2-(hydroxymethyl)-1,3- | 353.1 | 353.0 | (400 MHz, |
| | benzothiazol-5-yl]-4- | | | METHANOL-D4) δ |
| | (trifluoromethyl)benzamide | | | ppm 4.94 (s, 2H) |
| | | | | 7.71 (dd, J = 8.69, 2.05 Hz, 1H) |
| | | | | 7.82 (d, J = 8.20 Hz, 2H) |
| | | | | 7.95 (d, J = 8.20 Hz, 1H) |
| | | | | 8.10 (d, J = 8.01 Hz, 2H) |
| | | | | 8.41 (d, J = 1.95 Hz, 1H) |
| 26 | 2,3-difluoro-N-[2-(hydroxymethyl)- | 389.0 | 388.8 | (400 MHz, DMSO-D6) |
| | 1,3-benzothiazol-5-yl]-4- | | | δ ppm 4.82 (s, 2H), |
| | (trifluoromethyl)benzamide | | | 6.24 (s, 1H), 7.62 (dd, |
| | | | | J = 8.69, 1.85 Hz, 1H), |
| | | | | 7.68-7.78 (m, 2H), |
| | | | | 8.03 (d, J = 8.59 Hz, 1H), |
| | | | | 8.34 (d, J = 1.76 Hz, 1H), |
| | | | | 10.87 (s, 1H) |
| 27 | 4-fluoro-N-[2-(hydroxymethyl)- | 371.0 | 370.8 | (400 MHz, |
| | 1,3-benzothiazol-5-yl]-3- | | | METHANOL-D4) δ |
| | (trifluoromethyl)benzamide | | | ppm 4.96 (s, 2H) |
| | | | | 7.51 (t, J = 9.47 Hz, 1H) |
| | | | | 7.73 (dd, J = 8.79, 1.95 Hz, 1H) |
| | | | | 7.97 (d, J = 8.79 Hz, 1H) |
| | | | | 8.28-8.33 (m, 1H) |
| | | | | 8.35 (d, J = 6.83 Hz, 1H) |
| | | | | 8.41 (d, J = 1.76 Hz, 1H) |
| 28 | 4-(difluoromethoxy)-N-[2- | 351.1 | 351.0 | (400 MHz, |
| | (hydroxymethyl)-1,3-benzothiazol- | | | METHANOL-D4) δ |
| | 5-yl]benzamide | | | ppm 4.95 (s, 2H) |
| | | | | 6.98 (t, J = 73.52 Hz, 1H) |
| | | | | 7.27 (d, J = 8.59 Hz, 2H) |
| | | | | 7.71 (dd, J = 8.69, 2.05 Hz, 1H) |
| | | | | 7.95 (d, J = 8.79 Hz, 1H) |
| | | | | 8.02 (d, J = 8.79 Hz, 2H) |
| | | | | 8.40 (d, J = 1.76 Hz, 1H) |
| 29 | N-[2-(hydroxymethyl)-1,3- | 384.3 | 384.0 | 1H NMR (400 MHz, |
| | benzothiazol-5-yl]-6-(2,2,2- | | | DMSO-D6) δ ppm |
| | trifluoroethoxy)nicotinamide | | | 4.82 (s, 2H), 5.07 (q, J = 9.11 Hz, |
| | | | | 2H), 7.12 (d, J = 8.59 Hz, |
| | | | | 1H), 7.70 (dd, |
| | | | | J = 8.59, 1.95 Hz, 1H), |
| | | | | 8.00 (d, J = 8.59 Hz, 1H), |
| | | | | 8.32 (dd, J = 8.69, 2.44 Hz, |
| | | | | 1H), 8.39 (d, J = 1.76 Hz, |
| | | | | 1H), 8.79 (d, J = 2.15 Hz, |
| | | | | 1H), 10.47 (s, 1H) |
| 30 | N-[2-(hydroxymethyl)-1,3- | 357.3 | 357.0 | 1H NMR (400 MHz, |
| | benzothiazol-5-yl]-1-methyl-3- | | | DMSO-D6) δ ppm |
| | (trifluoromethyl)-1H-pyrazole-5 | | | 1.40-1.75 (m, 6H), |
| | carboxamide | | | 1.77-1.97 (m, 2H), |
| | | | | 2.11-2.30 (m, 1H), |
| | | | | 2.48-2.63 (m, 1H), 4.72 (s, 2H), |
| | | | | 5.93-6.26 (m, 1H), |
| | | | | 7.45 (d, J = 8.79 Hz, 1H), |
| | | | | 7.84 (d, J = 8.59 Hz, 1H), |
| | | | | 8.16 (s, 1H), 9.90 (s, 1H) |
| 31 | N-[2-(hydroxymethyl)-1,3- | 421.0 | 420.8 | 1H NMR (400 MHz, |
| | benzothiazol-5-yl]-2,4- | | | METHANOL-D4) δ |
| | bis(trifluoromethyl)benzamide | | | ppm 4.96 (s, 2H) |
| | | | | 7.64 (dd, J = 8.79, 2.15 Hz, 1H) |
| | | | | 7.85-8.03 (m, 2H) |
| | | | | 8.03-8.16 (m, 2H) |
| | | | | 8.36 (d, J = 1.95 Hz, 1H) |

### Example 32

### 4-tert-butyl-N-[2-(hydroxymethyl)-1,3-benzothiazol-5-yl]-2,6-dimethylbenzamide

P214 (431 mg, 0.757 mmol), was suspended in carbon tetrachloride (7.60 mL) and the amine (200 mg, 0.757 mmol) was added followed by 2,6-lutidine (88.0 μL, 0.757 mmol). After 40 minutes, 2,6-dimethyl-4-tert-butylcarboxylic acid (156 mg, 0.757 mg) in solution in DCM (7.60 mL) was added. The mixture was heated under gentle reflux for 16 hours. After cooling the reaction was quenched with 1M HCl. The organic phase was washed with an aqueous solution of sodium carbonate, brine, dried with anhydrous sodium sulfate and concentrated under reduced pressure to yield the amide. The amide product was mixed with aqueous 1M NaOH (10.0 mL) and THF (10.0 mL). The mixture was stirred for 30 minutes, the organic phase was separated and evaporated to dryness. The product was purified by Gilson HPLC (Luna 15 u, C18 (2), 250 mm×21.2 mm) eluting with mixtures of MeCN and H₂O containing 0.1% TFA to yield the product (24 mg, 0.065 mmol, 9.0%). ¹H NMR (400 MHz, METHANOL-D4) δ ppm 1.31 (s, 9H) 2.37 (s, 6 H) 4.95 (s, 2 H) 7.15 (s, 2 H) 7.64 (d, J=8.59 Hz, 1 H) 7.96 (d, J=8.79 Hz, 1 H) 8.44 (s, 1 H). MS [MH+] calc. 369.2, found 369.0. Anal found C 64.52%, H 6.13%, N 6.80%.

### Example 33

### N-[2-(hydroxymethyl)-1,3-benzothiazol-5-yl]-4-methoxy-2-methylbenzamide

Allyl(5-amino-1,3-benzothiazol-2-yl)methyl carbonate (500 mg, 1.89 mmol) was coupled to 4-methoxy-2-methylbenzoic acid (377 mg, 2.27 mmol) with EDC (434 mg, 2.27 mmol) and DMAP (277 mg, 2.27 mmol) in anhydrous DMF (10.0 mL). The reagents were stirred together for 18 hours at room temperature. The reaction mixture was dissolved in EtOAc then washed with, distilled water, HCl 1N, NaOH 1N, then distilled water, dried on anhydrous sodium sulfate, filtered and concentrated. The resulting residue was triturated in methanol and filtered to give the title compound (620 mg, 79%). ¹H NMR (400 MHz, CHLOROFORM-D) δ ppm 2.54 (s, 3 H) 3.85 (s, 3 H) 4.68-4.75 (m, 2 H) 5.28-5.34 (m, 1 H) 5.37-5.46 (m, 1 H) 5.55 (s, 2 H) 5.87-6.08 (m, 1 H) 6.72-6.86 (m, 2 H) 7.51 (d, J=8.20 Hz, 1 H) 7.64 (s, 1 H) 7.80 (d, 1 H) 7.86 (d, 1 H) 8.20 (s, 1 H).

A fraction of this product (500 mg, 1.21 mmol) was dissolved in THF (20 mL), MeOH (20 mL), water (2.0 mL) and solid sodium hydroxide (400 mg, 10 mmol) was added. The reagents were stirred together for 1 hour at room temperature. EtOAc was added and the resulting organic layer was washed with distilled water, dried over anhydrous sodium sulphate, filtered and concentrated. The resulting residue was triturated in EtOAc to give the desired product (200 mg, 50%). LC ret. time 1.46 minutes (Column: Phenomonex Polar, Gradient: 10-95% B, Flow rate: 1.75 mL/min, Column temperature: 40° C., Mobile phase: A −0.1% TFA in H₂O, B −0.1% TFA in MeCN). ¹H NMR (400 MHz, DMSO-D6) δ ppm 1.66 (s, 3 H) 3.02 (s, 3 H) 4.14 (s, 2 H) 5.92-6.15 (m, 2 H) 6.67 (d, J=8.01 Hz, 1 H) 6.85 (d, J=8.79 Hz, 1 H) 7.12 (d, J=8.79 Hz, 1 H) 7.58 (s, 1 H). MS [MH+] calc. 357.1 found 357.0.

### Example 34

### 2-but-3-en-1-yl-N-[2-(hydroxymethyl)-1,3-benzothiazol-5-yl]-4-methoxybenzamide

Allyl(5-amino-1,3-benzothiazol-2-yl)methyl carbonate (300 mg, 1.13 mmol) was coupled to 2-but-3-en-1-yl-4-methoxy-2-methylbenzoic acid (280 mg, 1.36 mmol) with EDC (260 mg, 1.36 mmol) and DMAP (164 mg, 1.36 mmol) in anhydrous DMF (1.0 mL)+DCM (1.0 mL). The reagents were stirred together for 18 hours at room temperature. To this reaction mixture was added water (1.0 mL) and solid sodium hydroxide (202 mg, 5 mmol) was added. The reagents were stirred together for 1 hour at room temperature. The reaction was monitered By LCMS. EtOAc was added and the resulting organic layer was washed with distilled water, dried over anhydrous sodium sulphate, filtered and concentrated. The resulting residue was purified on silicagel (60% EtoAc:40% heptane) to give 310 mg (74%) of the desire product 2-but-3-en-1-yl-N-[2-(hydroxymethyl)-1,3-benzothiazol-5-yl]-4-methoxybenzamide ¹H NMR (600 MHz, DMSO-D6) δ ppm 2.40 (q, J=7.43 Hz, 2 H) 2.86-3.05 (m, 2 H) 3.85 (s, 3 H) 4.92-5.09 (m, 4 H) 5.85 (dd, J=17.02, 10.56 Hz, 1 H) 6.78-7.02 (m, 2 H) 7.49 (d, J=7.63 Hz, 1 H) 7.67 (d, J=8.22 Hz, 1 H) 7.96 (d, J=8.22 Hz, 1 H) 8.40 (s, 1 H). MS [MH+] calc. 3.69.1 found 369.0

### Example 35

### 2-butyl-N-[2-(hydroxymethyl)-1,3-benzothiazol-5-yl]-4-methoxybenzamide

Allyl(5-amino-1,3-benzothiazol-2-yl)methyl carbonate (100 mg, 0.27 mmol) was reduced with 5% palladium on carbon (10 g) in methanol (50 mL). This mixture was shaken for 30 min. under 30 PSI of hydrogene. The reaction mixture was filtered and concentrated. The resulting residue was purified by reverse phase chromatography (on a Luna C18 Phenomenex reverse phase column) to give the desired product (27 mg, 26%) 2-butyl-N-[2-(hydroxymethyl)-1,3-benzothiazol-5-yl]-4-methoxybenzamide. 1H NMR (400 MHz, CHLOROFORM-D) δ ppm 0.88 (t, J=7.32 Hz, 3 H) 1.24-1.44 (m, 2 H) 1.48-1.69 (m, 2 H) 2.65-2.98 (m, 2 H) 3.82 (s, 3 H) 5.00 (s, 2 H) 6.54 (s, 1 H) 6.71 (dd, J=8.50, 2.44 Hz, 1 H) 6.77 (d, J=2.54 Hz, 1 H) 7.43 (d, J=8.40 Hz, 1 H) 7.75 (s, 2 H) 7.94-8.24 (m, 2 H). MS [MH+] calc. 371.1 found 371.0.

### Example 36

### N-[2-(hydroxymethyl)-1,3-benzothiazol-5-yl]-2-(methylamino)-6-(trifluoromethyl)-nicotinamide

The crude intermediate 5, 2-chloro-N-[2-(hydroxymethyl)-1,3-benzothiazol-5-yl]-6-(trifluoromethyl)-nicotinamide in THF (1.0 mmol in 5.0 mL) was stirred at room temperature while methylamine solution (4.0 mL, 1.0M in THF) was added via syringe. The contents were stirred for 16 hours, then the solvent was removed by rotary evaporator to yield a residue that was purified by reverse phase liquid chromatography to give the title compound as the TFA salt. Yield=51 mg, 10%. 1H NMR (400 MHz, DMSO-D6) δ ppm 2.87 (d, J=4.69 Hz, 3 H), 4.81 (d, J=5.47 Hz, 2 H), 6.24-6.32 (m, 1 H), 7.03 (d, J=7.62 Hz, 1 H), 7.65 (dd, J=8.69, 1.86 Hz, 1 H), 7.84 (d, J=4.69 Hz, 1 H), 8.00 (d, J=8.79 Hz, 1 H), 8.15 (d, J=7.62 Hz, 1 H), 8.31 (d, J=1.76 Hz, 1 H), 10.58 (s, 1 H). MS [MH+] calc. 382.4, found 383.0.

### Example 37

### N-[2-(hydroxymethyl)-1,3-benzothiazol-5-yl-2-(propylamino)-6-(tritluoromethyl)-nicotinamide

The crude intermediate 5, 2-chloro-N-[2-(hydroxymethyl)-1,3-benzothiazol-5-yl]-6-(trifluoromethyl)-nicotinamide in THE (0.5 mmol in 5.0 mL) was stirred at room temperature while n-propylamine (1.0 mL, 16.8 mmol) was added via syringe. The contents were stirred for 16 hours, then the solvent was removed by rotary evaporator to yield a residue that was purified by reverse phase liquid chromatography to give the title compound as the TFA salt. Yield=43 mg, 16%. 1H NMR (400 MHz, DMSO-D6) δ ppm 0.87 (t, J=7.42 Hz, 3 H) 1.50-1.61 (m, Hz, 2 H) 3.25-3.38 (m, 2 H) 4.82 (d, J=5.27 Hz, 2 H) 6.23 (t, J=5.86 Hz, 1 H) 7.04 (d, J=7.81 Hz, 1 H) 7.98-8.07 (m, 2 H) 8.22 (d, J=7.81 Hz, 1 H) 8.30 (d, J=1.76 Hz, 1 H) 10.57 (s, 1 H). MS [MH+] calc. 410.4, found 411.0.

### Example 38

### 2-(dimethylamino)-N-[2-(hydroxymethyl)-1,3-benzothiazol-5-yl]-6-(trifluoromethyl)-nicotinamide

The crude intermediate 5, 2-chloro-N-[2-(hydroxymethyl)-1,3-benzothiazol-5-yl]-6-(trifluoromethyl)-nicotinamide in THF (0.5 mmol in 5.0 mL) was stirred at room temperature while a solution of dimethylamine (2.0 mL, 1.0 M in THF) was added via syringe. The contents were stirred for 16 hours, then the solvent was removed by rotary evaporator to yield a residue that was purified by reverse phase liquid chromatography to give the title compound as the TFA salt. Yield=37 mg, 15%. 1H NMR (400 MHz, DMSO-D6) δ ppm 3.00 (s, 6 H), 4.81 (s, 2 H), 7.10 (d, J=7.62 Hz, 1 H), 7.61 (dd, J=8.69, 1.86 Hz, 1 H), 7.89 (d, J=7.42 Hz, 1 H), 7.99 (d, J=8.79 Hz, 1 H), 8.34 (d, J=1.76 Hz, 1 H), 10.70 (s, 1 H). MS [MH+] calc. 396.4, found 397.0.

### Example 39

### 2-ethyl-N-[2-(hydroxymethyl)-1,3-benzothiazol-5-yl]-4-isopropoxybenzamide

A mixture of 4-isopropoxy-2-methylbenzoic acid and 2-ethyl-4-isopropoxybenzoic acid (˜9:1, 0.735 g, 3.79 mmol, 1 equiv) was dissolved in 50 mL of dichloromethane. EDC (0.871 g, 4.55 mmol, 1.2 equiv), DMAP (0.926 g, 7.58 mmol, 2 equiv) and allyl(5-amino-1,3-benzothiazol-2-yl)methyl carbonate (1 g, 3.79 mmol, 1 equiv) were added, and the mixture stirred at room temperature for 6 h. The solvent was evaporated, the residue was dissolved in 20 mL of THF/12 mL of NaOH 2M, and the mixture stirred overnight at room temperature. After dilution with diethylether, phases were separated, the organic phase was dried over MgSO₄, filtered and evaporated to dryness. The yellow residue contained some DMAP. It was dissolved in EtOAc and washed with HCl 1M, affording 962 mg of a mixture of N-[2-(hydroxymethyl)-1,3-benzothiazol-5-yl]-4-isopropoxy-2-methylbenzamide and 2-ethyl-N-[2-(hydroxymethyl)-1,3-benzothiazol-5-yl]-4-isopropoxybenzamide. Those were separated by reverse-phase HPLC (MeCN/H₂O 40:60 to 60:40 over 1 h), giving 673 mg of N-[2-(hydroxymethyl)-1,3-benzothiazol-5-yl]-4-isopropoxy-2-methylbenzamide and 21 mg of 2-ethyl-N-[2-(hydroxymethyl)-1,3-benzothiazol-5-yl]-4-isopropoxybenzamid as their TFA salts. ¹H NMR (400 MHz, CD₃OD, δ ppm): 1.24 (t, J=7.62 Hz, 3 H); 1.32 (d, J=6.05 Hz, 6 H); 2.83 (q, J=7.42 Hz, 2 H); 4.67 (septet, J=6.06 Hz, 1 H); 4.95 (s, 2 H); 6.78-6.87 (m, 2 H); 7.43 (d, J=8.20 Hz, 1 H); 7.65 (d, J=8.79 Hz, 1 H); 7.94 (d, J=8.59 Hz, 1 H); 8.38 (s, 1 H); 10.29 (s, 1 H). LC-MS: MS [MH+] calc. 371.4, found 371.0

### Example 40

### N-[4-fluoro-2-(hydroxymethyl)-1,3-benzothiazol-5-yl]-4-isopropoxy-2-methylbenzamide

Allyl(5-amino-4-fluoro-1,3-benzothiazol-2-yl)methyl carbonate (143 mg, 0.507 mmol) was coupled to 4-isopropoxy-2-methylbenzoic acid (98.4 mg, 0.507 mmol) with EDC (100 mg, 0.520 mmol) and DMAP (64.0 mg, 0.520 mmol) in DCM (10.0 mL). The product was purified by flash chromatography on silica gel eluting with mixtures of hexanes and EtOAc (9:1 to 1:1) (68.0 mg, 0.148 mmol, 29%). The product was dissolved in THF (3.00 mL) and aqueous NaOH (3.00 mL, 1 N) was added. The mixture was stirred for 30 minutes and then evaporated to dryness. The product was purified by flash chromatography on silica gel eluting with mixtures of hexanes and EtOAc (9:1 to 1:1) (26.0 mg, 0.0570 mmol, 38.0%). ¹H NMR (400 MHz, CHLOROFORM-D) δ ppm 1.39 (d, J=6.05 Hz, 6 H) 2.55 (s, 3 H) 4.62 (ddd, J=17.77, 11.91, 5.86 Hz, 1 H) 5.11 (s, 2 H) 6.76-6.82 (m, 2 H) 7.56 (d, J=8.40 Hz, 1 H) 7.68 (dd, J=8.79, 1.17 Hz, 1 H) 8.45-8.58 (m, 1 H); MS [M+H] calcd. 375.1, found 375.0.

### Example 41

### N-[4-fluoro-2-(hydroxymethyl)-1,3-benzothiazol-5-yl]-2-methyl-6-(trifluoromethyl)nicotinamide

To a solution of allyl(5-amino-4-fluoro-1,3-benzothiazol-2-yl)methyl carbonate (130 mg, 0.33 mmol), 2-methyl-6-(trifluoromethyl)nicotinic acid (80 mg, 0.39 mmol) and DMAP (96 mg, 0.79 mmol) in a 1:1 mixture of DMF/dichloromethane (10 mL) was added EDC (151 mg, 0.79 mmol). The reaction was stirred overnight at room temperature under N₂. The next day, sodium hydroxide pellets (200 mg) and water (4 mL) were added and the reaction was stirred vigourously for 2 hours. The reaction mixture was diluted with dichloromethane and washed with water. The aqueous layer was extracted with two portions of dichloromethane and the combined organics were dried (MgSO₄), filtered and concentrated. The residue was purified by reverse phase chromatography using a LUNA C-18 column (250×21.20 mm, 15 μm particle size), gradient 5-75% B in 40 min, flow rate 40 mL/min, 20° C., A: 0.1% TFA in H₂O, B: 0.1% TFA in CH₃CN to give the product as a colourless solid (50 mg, 24%). ¹H NMR (400 MHz, DMSO-d6) δ ppm 2.68 (s, 3H), 4.90 (s, 2H), 7.72-7.79 (m, 1H), 7.88 (d, J=7.81 Hz, 1H), 7.94 (d, J=8.59 Hz, 1H), 8.22 (d, J =8.01 Hz, 1H), 10.61-10.68 (m, 1H). M.S. (calcd): 386.0 (MH⁺), M.S (found): 386.0 (MH⁺). HPLC: k′ 3.94; Purity: >99% (215 nm), >97% (254 nm), >99% (280 nm). Conditions: Zorbax C-18, gradient 10-95% B in 25 min, flow rate 1 mL/min, 25° C., A: 0.1% TFA in H₂O, B: 0.1% TFA in CH₃CN. Found: C, 50.18; H, 3.01; N, 10.70. C₁₆H₁₁F₄N₃O₂S has C, 49.87; H, 2.88; N, 10.90%.

### Example 42

### N-[4-fluoro-2-(hydroxymethyl)-1,3-benzothiazol-5-yl]-6-(trifluoromethyl)nicotinamide

Using the same procedure as Example 1 with allyl(5-amino-4-fluoro-1,3-benzothiazol-2-yl)methyl carbonate (400 mg, 1.01 mmol) and 6-(trifluoromethyl)nicotinic acid (231 mg, 1.21 mmol) afforded the title compound as a pale yellow solid (210 mg, 56%). ¹H NMR (400 MHz, DMSO-d6) δ ppm 4.98 (s, 2H), 7.76 (dd, J=8.59, 6.25 Hz, 1H), 7.84 (dd, J=8.79, 1.17 Hz, 1H), 8.00 (dd, J=8.20, 0.59 Hz, 1H), 8.58 (dd, J=8.20, 1.76 Hz, 1H), 9.24-9.29 (m, 1H). M.S. (calcd): 372.0 (MH⁺), M.S (found): 372.0 (MH⁺). HPLC: k′ 3.74; Purity: >97% (215 nm), >96% (254 nm), >99% (280 nm). Conditions: Zorbax C-18, gradient 10-95% B in 25 min, flow rate 1 mL/min, 25° C., A: 0.1% TFA in H₂O, B: 0.1% TFA in CH₃CN. Found: C, 48.40; H, 2.12; N, 10.97. C₁₅H₉F₄N₃O₂S×0.05 CF₃CO₂H has C, 48.11; H, 2.42; N, 11.15%.

### Example 43

### N-[4-fluoro-2-(hydroxymethyl)-1,3-benzothiazol-5-yl]-6-(2,2,2-trifluoroethoxy)nicotinamide

Using the same procedure as Example 1 with allyl(5-amino-4-fluoro-1,3-benzothiazol-2-yl)methyl carbonate (418 mg, 1.06 mmol) and 6-(2,2,2-trifluoroethoxy)nicotinic acid (280 mg, 1.27 mmol) afforded the title compound as a colourless solid (89 mg, 21%). ¹H NMR (400 MHz, DMSO-d6) δ ppm 4.87-4.91 (m, 1H), 5.10 (q, J=9.19 Hz, 2H), 6.35-6.40 (m, 1H), 7.15 (d, J=8.79 Hz, 1H), 7.56 (dd, J=8.20, 6.84 Hz, 1H), 7.91 (d, J=8.59 Hz, 1H), 8.36 (dd, J=8.59, 2.15 Hz, 1H), 8.85 (s, 1H), 10.42 (s, 2H). M.S. (calcd): 402.1 (MH⁺), M.S (found): 401.7 (MH⁺). HPLC: k′ 4.42; Purity: >98% (215 nm), >96% (254 nm), >97% (280 nm). Conditions: Zorbax C-18, gradient 10-95% B in 25 min, flow rate 1 mL/min, 25° C., A: 0.1% TFA in H₂O, B: 0.1% TFA in CH₃CN. Found: C, 47.73; H, 3.00; N, 10.26. C₁₆H₁₁F₄N₃O₃S×0.15 H₂O has C, 47.56; H, 2.82; N, 10.40%.

### Example 44

### N-[4-fluoro-2-(hydroxymethyl)-1,3-benzothiazol-5-yl]-1-methyl-3-(trifluoromethyl)-1H-pyrazole-5-carboxamide

Using the same procedure as Example 1 with allyl(5-amino-4-fluoro-1,3-benzothiazol-2-yl)methyl carbonate TFA salt (250 mg, 0.661 mmol) and 1-methyl-3-(trifluoromethyl)-1H-pyrazole-5-carboxylic acid (193 mg, 0.992 mmol) afforded the title compound 1H NMR (400 MHz, DMSO-D6) δ ppm 4.13 (s, 3 H), 4.86 (s, 2 H), 6.35 (s, 1 H), 7.47-7.57 (m, 2 H), 7.90 (d, J=8.59 Hz, 1 H), 10.51 (s, 1 H). M.S. (calcd): 375.1 (MH⁺), M.S (found): 374.8 (MH⁺).

Compounds in the following examples were synthesized according to the amide bond-forming procedures described in the example 1 or 2 starting from an appropriate and aromatic amine (corresponding to intermediate 9) or synthesized according to the procedures described in PCT/SE2004/000635, and an appropriately substituted commercially available aromatic acid or an aromatic acyl chloride (unless stated otherwise these were commercially available). The amide bond-forming procedures were followed by the deprotection as described in Example 2.

| Example | | MS | MS | |
|---|---|---|---|---|
| Nr | Name | Calc. | found | 1H NMR |
| 45 | 4-(dimethylamino)-N-[2- | 418.1 | 418.0 | (400 MHz, DMSO-D6) |
| | (hydroxymethyl)-1,3-benzothiazol- | | | δ ppm 2.80 (s, 6H) |
| | 5-yl]-3,5-dinitrobenzamide | | | 4.83 (d, J = 6.05 Hz, 2H) |
| | | | | 6.24 (t, J = 5.96 Hz, 1H) |
| | | | | 7.71 (dd, J = 8.79, 2.15 Hz, 1H) |
| | | | | 8.02 (d, J = 8.79 Hz, 1H) |
| | | | | 8.37 (d, J = 1.95 Hz, 1H) |
| | | | | 8.65 (s, 2H) 10.60 (s, |
| | | | | 1H) |
| 46* | N-[2-(hydroxymethyl)-1,3- | 367.1 | 367.0 | (400 MHz, |
| | benzothiazol-5-yl]-2-methyl-5- | | | METHANOL-D4) |
| | (trifluoromethyl)benzamide | | | δ ppm 2.54 (s, 3H) |
| | | | | 4.87 (s, 2H) 7.52 (d, J = 8.01 Hz, |
| | | | | 1H) 7.64-7.74 (m, |
| | | | | 2H) 7.79 (d, J = 0.78 Hz, |
| | | | | 1H) 7.96 (d, J = 8.59 Hz, |
| | | | | 1H) 8.42 (d, J = 1.95 Hz, |
| | | | | 1H) |
| 47** | 6-(4-fluorophenyl)-N-[2- | 394.1 | 394.0 | (400 MHz, DMSO-D6) |
| | (hydroxymethyl)-1,3-benzothiazol- | | | δ ppm 2.66 (s, 3H) |
| | 5-yl]-2-methylnicotinamide | | | 4.86 (d, J = 5.86 Hz, 2H) |
| | | | | 6.26 (t, J = 5.96 Hz, 1H) |
| | | | | 7.34 (t, J = 17.77, 8.79 Hz, 2H) |
| | | | | 7.71 (dd, J = 8.69, 1.86 Hz, |
| | | | | 1H) 7.94 (d, J = 8.20 Hz, |
| | | | | 1H) 8.03 (t, J = 8.10 Hz, |
| | | | | 2H) 8.20 (dd, |
| | | | | J = 8.98, 5.47 Hz, 2H) |
| | | | | 8.43 (d, J = 1.76 Hz, 1H) |
| | | | | 10.66 (s, 1H) |
| 48 | N-[2-(hydroxymethyl)-1,3- | 311.1 | 311.0 | (600 MHz, MeOD) δ |
| | benzothiazol-5-yl]-4- | | | ppm 4.98 (s, 1H) |
| | vinylbenzamide | | | 5.46 (d, J = 11.01 Hz, 1H) |
| | | | | 6.02 (d, J = 17.67 Hz, 1H) |
| | | | | 6.91 (dd, J = 17.41, |
| | | | | 11.01 Hz, 1H) 7.67 (d, |
| | | | | J = 7.42 Hz, 2H) 7.81 (d, |
| | | | | J = 8.45 Hz, 1H) |
| | | | | 7.92-8.11 (m, J = 6.91, 6.91 Hz, |
| | | | | 3H) 8.50 (s, 1H) |
| 49 | 4-ethynyl-N-[2-(hydroxymethyl)- | 309.1 | 309.0 | (600 MHz, |
| | 1,3-benzothiazol-5-yl]benzamide | | | CHLOROFORM-D) d |
| | | | | ppm 3.80 (s, 1H) |
| | | | | 4.97 (s, 2H) 7.70 (d, J = 8.31 Hz, |
| | | | | 2H) 7.81 (dd, |
| | | | | J = 8.69, 1.89 Hz, 1H) |
| | | | | 7.97-8.11 (m, 3H) |
| | | | | 8.50 (d, J = 1.51 Hz, 1H) |
| 50 | 4-bromo-N-[2-(hydroxymethyl)- | 407.0 | 406.7 | 1H NMR (600 MHz, |
| | 1,3-benzothiazol-5-yl]-2- | | | MeOD) d ppm 3.25 (d, |
| | (methoxymethyl)benzamide | | | J = 1.28 Hz, 3H) 4.51 (s, |
| | | | | 2H) 4.81 (s, 2H) |
| | | | | 7.39 (d, J = 7.94 Hz, 1H) |
| | | | | 7.45 (d, J = 8.19 Hz, 1H) |
| | | | | 7.50 (d, J = 8.70 Hz, 1H) |
| | | | | 7.57 (s, 1H) 7.80 (d, J = 8.45 Hz, |
| | | | | 1H) 8.25 (s, 1H) |
| 51 | 1-ethyl-N-[2-(hydroxymethyl)-1,3- | 352.1 | 352.0 | (400 MHz, DMSO-D6) |
| | benzothiazol-5-yl]-1H-indole-3- | | | d ppm 1.45 (t, J = 7.23 Hz, |
| | carboxamide | | | 3H) 4.30 (q, J = 7.23 Hz, |
| | | | | 2H) 4.85 (s, 2H) |
| | | | | 6.25 (br s, 1H) |
| | | | | 7.15-7.28 (m, 2H) 7.58 (d, |
| | | | | J = 8.20 Hz, 1H) |
| | | | | 7.76 (dd, J = 8.79, 1.95 Hz, 1H) |
| | | | | 7.99 (d, J = 8.79 Hz, 1H) |
| | | | | 8.22 (d, J = 7.62 Hz, 1H) |
| | | | | 8.36 (s, 1H) 8.43 (d, |
| | | | | J = 1.95 Hz, 1H) 9.93 (s, |
| | | | | 1H) |
| 52 | N-[2-(hydroxymethyl)-1,3- | 339.1 | 339.0 | (400 MHz, |
| | benzothiazol-5-yl]-5,6,7,8- | | | METHANOL-D4) δ |
| | tetrahydronaphthalene-2- | | | ppm 1.72-1.90 (m, 4H) |
| | carboxamide | | | 2.73-2.96 (m, 4H) |
| | | | | 4.95 (s, 2H) 7.18 (d, J = 7.62 Hz, |
| | | | | 1H) 7.56-7.79 (m, |
| | | | | 3H) 7.93 (d, J = 8.79 Hz, |
| | | | | 1H) 8.39 (dd, J = 3.51, |
| | | | | 1.95 Hz, 1H) |
| 53 | 2-bromo-N-[2-(hydroxymethyl)- | 383.9 | 383.7 | (400 MHz, DMSO-D6) |
| | 1,3-benzothiazol-5-yl]-4-methyl- | | | d ppm 2.59 (s, 3H) |
| | 1,3-thiazole-5-carboxamide | | | 4.85 (d, J = 6.0 Hz, 2H) |
| | | | | 6.26 (t, J = 6.0 Hz, 1H) |
| | | | | 7.63 (dd, J = 8.6, 1.8 Hz, 1H) |
| | | | | 8.03 (d, J = 8.6 Hz, 1H) |
| | | | | 8.28 (d, J = 1.8 Hz, 1H) |
| | | | | 10.45 (s, 1H) |
| 54 | 4-chloro-N-[2-(hydroxymethyl)- | 333.0 | 333.0 | (400 MHz, |
| | 1,3-benzothiazol-5-yl]-2- | | | METHANOL-D4) δ |
| | methylbenzamide | | | ppm 2.45 (s, 3H) |
| | | | | 4.94 (s, 2H) 7.28-7.36 (m, 2H) |
| | | | | 7.48 (d, J = 8.20 Hz, 1H) |
| | | | | 7.65 (dd, J = 8.79, 1.76 Hz, |
| | | | | 1H) 7.94 (d, J = 8.79 Hz, |
| | | | | 1H) 8.40 (d, J = 1.56 Hz, |
| | | | | 1H) |

*Intermediate 10 was used as the acid.
**The corresponding acid preparation is reported in WO 2004/072069.

Pharmacology

1. hVR1 FLIPR (Fluorometric Image Plate Reader) Screening Assay

Transfected CHO cells, stably expressing hVR1 (15,000 cells/well) are seeded in 50 ul media in a black clear bottom 384 plate (Greiner) and grown in a humidified incubator (37° C., 2% CO₂), 24-30 hours prior to experiment.

Subsequently, the media is removed from the cell plate by inversion and 2 μM Fluo-4 is added using a multidrop (Labsystems). Following the 40 minutes dye incubation in the dark at 37° C. and 2% CO₂, the extracellular dye present is washed away using an EMBLA (Scatron), leaving the cells in 40 ul of assay buffer (1× HBSS, 10 mM D-Glucose, 1 mM CaCl₂, 10 mM HEPES, 10×7.5% NaHCO₃ and 2.5 mM Probenecid).

FLIPR Assay—IC₅₀ Determination Protocol

For IC₅₀ determinations the fluorescence is read using FLIPR filter 1 (em 520-545 nM). A cellular baseline recording is taken for 30 seconds, followed by a 20 μl addition of 10, titrated half-log concentrations of the test compound, yielding cellular concentration ranging from 3 μM to 0.1 nM. Data is collected every 2 seconds for a further 5 minutes prior to the addition of a VR1 agonist solution: either 50 nM solution of capsaicin or MES (2-[N-morpholino]ethanesulfonic acid) buffer (pH 5.2), by the FLIPR pipettor. The FLIPR continues to collect data for a further 4 minutes. Compounds having antagonistic properties against the hVR1 will inhibit the increase in intracellular calcium in response to the capsaicin addition. This consequently leading to a reduction in fluorescence signal and providing a reduced fluorescence reading, compared with no compound, buffer controls. Data is exported by the FLIPR program as a sum of fluorescence calculated under the curve upon the addition of capsaicin. Maximum inhibition, Hill slope and IC₅₀ data for each compound are generated. IC₅₀ data are given in Table 1 below.

2. DRGs were dissected out from adult Sprague Dawley rats (100-300 gr), and placed on ice in L15 Leibovitz medium. The ganglia were enzyme treated with Collagenase 80 U/ml+ Dispase 34 U/ml dissolved in DMEM +5% serum, overnight at 37° C. The next day, cells were triturated with fire polished pasteur pipettes, and seeded in the center of 58 mm diameter Nune cell dishes coated with Poly-D Lysine (1 mg/mL). The DRGs were cultured in a defined medium without foetal bovine serum, containing Dulbecco's MEM/NUT MIX F-12 (1:1) without L-glutamine but with pyridoxine, 6 mg/mL D(+)-Glucose, 100 μg/mL apo-transferrin, 1 mg/mL BSA, 20 μg/mL insulin, 2 mM L-glutamine, 50 IU/mL Penicillin, 50 μg/mL Streptomycin and 0.01 μg/mL NGF-7S.

When the cells had grown for 2 days up to 4 weeks, the experiments were done. Cells were chosen based on size and presence of neurites. Small cells with long processes were used for recording (most likely to be C neurons, with native VR1 receptors).

The cells were recorded with conventional whole cell voltage clamp patch clamp, using the following solutions (calcium ion free):

The extracellular solution comprised (in mM): NaCl 137, KCl 5, MgCl₂*H₂O 1.2, HEPES 10, Glucose 10, EGTA 5, Sucrose 50, pH to 7.4 with NaOH.

The intracellular solution comprised K-gluconate 140, NaCl 3, MgCl₂*H₂O 1.2, HEPES 10, EGTA 1, pH to 7.2 with KOH. When the cells were penetrated with suction, a puff of capsaicin (500 nM) was used to determine if the cell expressed VR1 receptor. If not, a new cell was chosen. If yes, then the compounds were added in increasing doses before the capsaicin pulse (500 nM), to determine an IC₅₀ value.

Male Sprague-Dawley rats (Charles River, St-Constant, Québec, Canada), weighing 200-210 g, were housed under standard conditions (light/dark cycle of 12 h; room temperature: 20° C.) with food and water ad libitum. The drug was administrated to three rats as a bolus injection into the tail vein at a dose level of 23.5 μmol/kg/2 ml. Blood samples (250-300 μl) were obtained from the tail into heparinized tubes (10 μl sodium heparin 1000 U/ml) at the following time points after drug administration 0.083, 0.5, 1, 2, 4, 6, 8, and 10 h. Each blood sample was centrifuged immediately (5 min, 3000×g) and the plasma was separated and stored at −80° C. until analysis.

Experiments were carried out according to a protocol approved by AstraZeneca R&D Montreal and in accordance with policies and guidelines of the Canadian Council on Animal Care.

Rat plasma samples were kept frozen at −80° C. until analysis. Plasma proteins were precipitated with an equal volume of acetonitrile containing 0.1% v/v formic acid, vortex-mixed, and centrifuged (10,000×g, 30 min., 4° C.).

Detection of the parent compound and its metabolites was performed on a Waters system (Waters, Canada) coupled with a triple quadrupole mass spectrometer with an ESI source (Quattro Micro™ API from Micromass, USA). The chromatographic separation was achieved on an ACE 3 C18 column (2.1 mm×50 mm, 3μ from Life Science, Canada) thermostated at 45° C. Samples were injected (10 μL) onto the column using a 2777 sample manager (Waters, Canada). The mobile phase consisted of 0.1% v/v formic acid in water (solvent A) and 0.1% v/v formic acid in acetonitrile (solvent B). A step-wise linear gradient was used at a flow rate of 0.75 ml/min starting at 5 min with 20% of solvent B and ending at 9 min with 95% of solvent B. Acquisition was performed by monitoring the MRM transition m/z 357→4282.9 in positive ionization mode. Capillary and cone voltage were set at 0.4 kV and 25 V respectively and the collision energy at 22V. Extracted ion chromatograms were integrated using the Quanlynx software package (Micromass, Canada). Detection of its two metabolites was performed on an HPLC 1100 series system (Agilent Technologies, Canada) with a single quadrupole mass spectrometer with an ESI source. The chromatographic separation was achieved on an Allure PFP propyl column (2.1 mm×50 mm, 5μ from Restec, Canada) thermostated at 45° C. Samples were injected (10 μL) onto the column using a PAL injector (CTC Analytics, USA). The mobile phase consisted of 0.1% v/v formic acid in water (solvent A) and 0.1% v/v formic acid in acetonitrile (solvent B). A step-wise linear gradient was used at a flow rate of 1 ml/min. starting at 0.5 min with 0% of solvent B and ending at 3 min with 90% of solvent B. Acquisition was performed in selected ion-monitoring (SIM) mode (m/z=223 and 181 for the 2 metabolites tested) in positive ionization mode. The nebulizer pressure was set at 60 p.s.i.g., while the drying gas (nitrogen) was delivered at a flow rate of 13 L/min. at a temperature of 350° C. Capillary voltage was set at 3.5 kV and the fragmentor (collision-induced dissociation cell) was set at 50 and 60 V for the metabolites. Extracted ion chromatograms were integrated using the HP ChemStation software package (Rev 10.01, Agilent technologies, Canada).

The standard curve was constructed with drug free rat plasma as matrix and using twelve calibration points covering 4 log units. The standard curve equation relating concentrations with peak area (y=ax²+bx+c) was obtained from quadratic fitting (Xlfit, ID Business Solution Limited, U.K.). The method was run in a non-GLP setting. The LLOQ was 1.22 nM for the parent and 2.44 for its two metabolites.

Metabolic Stability in Rat Hepatocytes

Cells source: in house fresh isolated rat hepatocytes from male sprague-Dawley.

1 μM of compound incubated in Krebs-Heinsleit buffer with 1×10⁶ cells in 37° C. for 0, 15, 30, 60 and 90 min. A total volume of each incubation was 100 μL containing 0.1% DMSO. The reaction was stopped by adding 100 μL acetonitrile.

After reaction was stopped. The sample was centrifuged at 1900 g for 5 min. Supernatant was transferred to a clean tube for analysis.

Metabolic Stability in Human Cryopreserved Hepatocytes

Cells source: of human cryopreserved hepatocytes pooled from 5 individuals of both genders supplied by In Vitro Technologies

1 μM of compound incubated in Krebs-Heinsleit buffer with 1×10⁶ cells in 37° C. for 0, 15, 30, 60 and 90 min. A total volume of each incubation was 100 μL containing 0.1% DMSO. The reaction was stopped by adding 100 μL acetonitrile

After reaction was stopped. The sample was centrifuged at 1900 g for 5 min. Supernatant was transferred to a clean tube for analysis.

List of Abbreviations

VR1 vanilloid receptor 1

IBS irritable bowel syndrome

IBD inflammatory bowel disease

GERD gastro-esophageal reflux disease

DRG Dorsal Root Ganglion

BSA Bovine Serum Albumin

HEPES 4-(2-Hydroxyethyl)piperazine-1-ethanesulfonic acid

EGTA Ethylene glycol-bis(2-aminoethylether)-N,N,N′,N′-tetraacetic acid

DMEM Dulbeccos Modified Eagle's Medium

Results

Typical IC₅₀ values as measured in the assays described above are 10 μM or less. In one aspect of the invention the IC₅₀ is below 500 nM. In another aspect of the invention the IC₅₀ is below 100 nM. In a further aspect of the invention the IC₅₀ is below 10 nM.

TABLE 1
Specimen results from the hVR1 FLIPR.

| | IC₅₀ nM |
|---|---|
| Example | Unless otherwise stated the |
| No. | agonist was capsaicin |
| 5 | 8 |
| 6 | 23 |
| 7 | 4 |
| 8 | 417* |
| 9 | 86 |
| 10 | 54 |
| 23 | 59 |
| 24 | 47 |
| 40 | 67 |
| 41 | 1947 |
| 42 | 904 |
| 43 | 489 |

*The agonist was a pH 5.2 buffer

TABLE 2
Low intrinsic clearances for examples 10, 23, 24 and 30.

| Example | | rClint hep |
|---|---|---|
| No. | Structure | (ul/min./mg) |
| 23 | 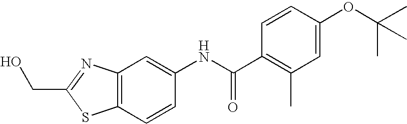 | 22 |
| 24 | 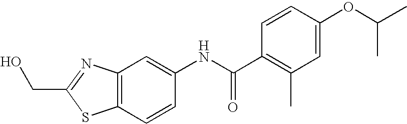 | 10 |
| 30 | 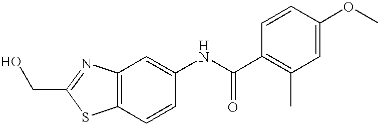 | 22 |
| 10 | 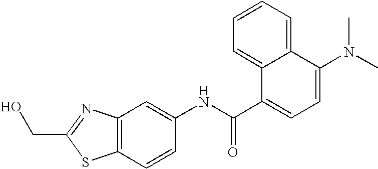 | 14 |
| 40 | 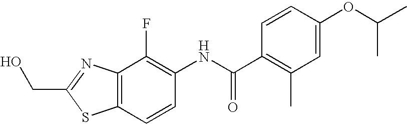 | 11.8 |

1. A compound of formula I 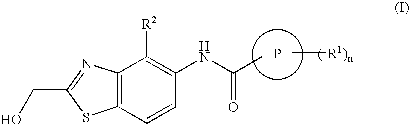
wherein:
ring P is C₆₋₁₀aryl, C₃₋₇cycloalkyl, C₅₋₆heteroaryl, which ring P may be fused with phenyl, C₅₋₆heteroaryl, C₃₋₇cycloalkyl or C₃₋₇heterocycloalkyl;
R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, OC₁₋₆haloalkyl, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆haloalkylOC₀₋₆haloalkyl, C₁₋₆alkylOC₀₋₆halolkyl, C₁₋₆alkylOC₀₋₆alkyl, OC₁₋₆alkyl, C₁₋₆alkylSC₀₋₆alkyl, C₁₋₆alkylSO, C₁₋₆alklylSO₂, C₁₋₆alkylNHCO or C₁₋₆alkylNC₀₋₆alkyl;
n is 1, 2, 3, 4 or 5; and
R² is H, F, or Cl,
or salts, solvates or solvated salts thereof.

2. The compound according to claim 1, with the proviso that the compound is not
3-fluoro-N-[2-(hydroxymethyl)-1,3-benzothiazol-5-yl]-4-(trifluoromethyl)benzamide,
4-tert-butoxy-N-[4-chloro-2-(hydroxymethyl)-1,3-benzothiazol-5-yl]benzamide,
4-(tert-Butoxymethyl)-N-[4-chloro-2-(hydroxymethyl)-1,3-benzothiazol-5-yl]benzamide,
4-Bromo-2-chloro-N-[2-(hydroxymethyl)-1,3-benzothiazol-5-yl]benzamide, or
4-Bromo-2-fluoro-N-[2-(hydroxymethyl)-1,3-benzothiazol-5-yl]benzamide.

3. The compound according to claim 1 wherein R² is H or Cl.

4. The compound according to claim 1 wherein R² is F.

5. The compound according to claim 1 wherein R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, C₁₋₆alkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl, C₁₋₆alkylSO, C₁₋₆alkylSO₂ and C₁₋₆alkylNHCO or C₁₋₆alkylNC₀₋₆alkyl.

6. The compound according to claim 1 wherein R² is F and R¹ is NO₂, NH₂, halo, N(C₁₋₆alkyl)₂, C₁₋₆alkyl, C₂₋₆alkenyl, C₂₋₆alkynyl, C₁₋₆haloalkyl, C₁₋₆haloalkylO, C₁₋₆alkylO, phenylC₀₋₆alkyl, C₅₋₆heteroarylC₀₋₆alkyl, C₃₋₇cycloalkylC₀₋₆alkyl, C₃₋₇heterocycloalkylC₀₋₆alkyl, C₁₋₆alkylOC₀₋₆alkyl, C₁₋₆alkylSC₀₋₆alkyl, C₁₋₆alkylSO, C₁₋₆alkylSO₂ and C₁₋₆alkylNHCO or C₁₋₆alkylNC₀₋₆alkyl.

7. Compounds selected from the group consisting of
3-tert-butoxy-N-[2-(hydroxymethyl)-1,3-benzothiazol-5-yl]benzamide,
4-(dimethylamino)-N-[2-(hydroxymethyl)-1,3-benzothiazol-5-yl]benzamide,
tert-butyl4-({[2-(hydroxymethyl)-1,3-benzothiazol-5-yl]amino}carbonyl)benzoate,
N,N-diethyl-N′-[2-(hydroxymethyl)-1,3-benzothiazol-5-yl]terephthalamide,
N-[2-(hydroxymethyl)-1,3-benzothiazol-5-yl]-4-(1,1,2,2-tetrafluoroethoxy)benzamide,
4-Cyclohexyl-N-[2-(hydroxymethyl)-1,3-benzothiazol-5-yl]benzamide,
3-Fluoro-N-[2-(hydroxymethyl)-1,3-benzothiazol-5-yl]-4-(trifluoromethyl)benzamide,
N-[2-(hydroxymethyl)-1,3-benzothiazol-5-yl]-6-(trifluoromethyl)nicotinamide,
N-[2-(hydroxymethyl)-1,3-benzothiazol-5-yl]-6-piperidin-1-ylnicotinamide,
4-(dimethylamino)-N-[2-(hydroxymethyl)-1,3-benzothiazol-5-yl]-1-naphthamide,
2-fluoro-N-[2-(hydroxymethyl)-1,3-benzothiazol-5-yl]-4-(trifluoromethyl)benzamide,
N-[2-(hydroxymethyl)-1,3-benzothiazol-5-yl]-2-methoxy-6-(trifluoromethyl)nicotinamide,
N-[2-(hydroxymethyl)-1,3-benzothiazol-5-yl]-2-methyl-6-(trifluoromethyl)nicotinamide,
3-fluoro-N-[2-(hydroxymethyl)-1,3-benzothiazol-5-yl]-2,4-dimethylbenzamide,
N-[2-(hydroxymethyl)-1,3-benzothiazol-5-yl]-2,4-dimethylbenzamide,
4-tert-butoxy-N-[4-chloro-2-(hydroxymethyl)-1,3-benzothiazol-5-yl]benzamide,
4-(tert-Butoxymethyl)-N-[4-chloro-2-(hydroxymethyl)-1,3-benzothiazol-5-yl]benzamide,
N-[4-chloro-2-(hydroxymethyl)-1,3-benzothiazol-5-yl]-4-isopropoxybenzamide,
3-tert-butoxy-N-[4-chloro-2-(hydroxymethyl)-1,3-benzothiazol-5-yl]benzamide,
tert-butyl4-({[4-chloro-2-(hydroxymethyl)-1,3-benzothiazol-5-yl]amino}carbonyl)benzoate,
4-Bromo-2-chloro-N-[2-(hydroxymethyl)-1,3-benzothiazol-5-yl]benzamide,
4-Bromo-2-fluoro-N-[2-(hydroxymethyl)-1,3-benzothiazol-5-yl]benzamide,
4-tert-butoxy-N-[2-(hydroxymethyl)-1,3-benzothiazol-5-yl]-2-methylbenzamide,
N-[2-(hydroxymethyl)-1,3-benzothiazol-5-yl]-4-isopropoxy-2-methylbenzamide,
N-[2-(hydroxymethyl)-1,3-benzothiazol-5-yl]-4-(trifluoromethyl)benzamide,
2,3-difluoro-N-[2-(hydroxymethyl)-1,3-benzothiazol-5-yl]-4-(trifluoromethyl)benzamide,
4-fluoro-N-[2-(hydroxymethyl)-1,3-benzothiazol-5-yl]-3-(trifluoromethyl)benzamide,
4-tert-butyl-N-[2-(hydroxymethyl)-1,3-benzothiazol-5-yl]-2,6-dimethylbenzamide,
N-[2-(hydroxymethyl)-1,3-benzothiazol-5-yl]-4-methoxy-2-methylbenzamide, and
4-(difluoromethoxy)-N-[2-(hydroxymethyl)-1,3-benzothiazol-5-yl]benzamide,
N-[4-fluoro-2-(hydroxymethyl)-1,3-benzothiazol-5-yl]-4-isopropoxy-2-methylbenzamide,
N-[2-(hydroxymethyl)-1,3-benzothiazol-5-yl]-6-(2,2,2-trifluoroethoxy)nicotinamide,
N-[2-(hydroxymethyl)-1,3-benzothiazol-5-yl]-1-methyl-3-(trifluoromethyl)-1H-pyrazole-5-carboxamide,
N-[2-(hydroxymethyl)-1,3-benzothiazol-5-yl]-2,4-bis(trifluoromethyl)benzamide,
2-but-3-en-1-yl-N-[2-(hydroxymethyl)-1,3-benzothiazol-5-yl]-4-methoxybenzamide,
N-[2-(hydroxymethyl)-1,3-benzothiazol-5-yl]-2-(methylamino)-6-(trifluoromethyl)-nicotinamide,
N-[2-(hydroxymethyl)-1,3-benzothiazol-5-yl]-2-(propylamino)-6-(trifluoromethyl)-nicotinamide,
2-(dimethylamino)-N-[2-(hydroxymethyl)-1,3-benzothiazol-5-yl]-6-(trifluoromethyl)-nicotinamide,
2-ethyl-N-[2-(hydroxymethyl)-1,3-benzothiazol-5-yl]-4-isopropoxybenzamide,
2-butyl-N-[2-(hydroxymethyl)-1,3-benzothiazol-5-yl]-4-methoxybenzamide,
N-[4-fluoro-2-(hydroxymethyl)-1,3-benzothiazol-5-yl]-2-methyl-6-(trifluoromethyl)nicotinamide,
N-[4-fluoro-2-(hydroxymethyl)-1,3-benzothiazol-5-yl]-6-trifluoromethyl)nicotinamide,
N-[4-fluoro-2-(hydroxymethyl)-1,3-benzothiazol-5-yl]-6-(2,2,2-trifluoroethoxy)nicotinamide,
N-[4-fluoro-2-(hydroxymethyl)-1,3-benzothiazol-5-yl]-1-methyl-3-(trifluoromethyl)-1H-pyrazole-5-carboxamide,
4-(dimethylamino)-N-[2-(hydroxymethyl)-1,3-benzothiazol-5-yl]-3,5-dinitrobenzamide,
N-[2-(hydroxymethyl)-1,3-benzothiazol-5-yl]-2-methyl-5-(trifluoromethyl)benzamide,
N-[2-(hydroxymethyl)-1,3-benzothiazol-5-yl]-5,6,7,8-tetrahydronaphthalene-2-carboxamide,
N-[2-(hydroxymethyl)-1,3-benzothiazol-5-yl]-4-vinylbenzamide,
4-ethynyl-N-[2-(hydroxymethyl)-1,3-benzothiazol-5-yl]benzamide,
4-bromo-N-[2-(hydroxymethyl)-1,3-benzothiazol-5-yl]-2-(methoxymethyl)benzamide,
1-ethyl-N-[2-(hydroxymethyl)-1,3-benzothiazol-5-yl]-1H-indole-3-carboxamide,
6-(4-fluorophenyl)-N-[2-(hydroxymethyl)-1,3-benzothiazol-5-yl]-2-methylnicotinamide,
2-bromo-N-[2-(hydroxymethyl)-1,3-benzothiazol-5-yl]-4-methyl-1,3-thiazole-5-carboxamide,
4-chloro-N-[2-(hydroxymethyl)-1,3-benzothiazol-5-yl]-2-methylbenzamide
and salts, solvates or solvated salts thereof.

8. A compound according to claim 7, with the proviso that the compound is not
3-fluoro-N-[2-(hydroxymethyl)-1,3-benzothiazol-5-yl]-4-(trifluoromethyl)benzamide,
4-tert-butoxy-N-[4-chloro-2-(hydroxymethyl)-1,3-benzothiazol-5-yl]benzamide,
4-(tert-Butoxymethyl)-N-[4-chloro-2-(hydroxymethyl)-1,3-benzothiazol-5-yl]benzamide,
4-Bromo-2-chloro-N-[2-(hydroxymethyl)-1,3-benzothiazol-5-yl]benzamide, or
4-Bromo-2-fluoro-N-[2-(hydroxymethyl)-1,3-benzothiazol-5-yl]benzamide.

9. A pharmaceutical composition comprising as active ingredient a therapeutically effective amount of the compound according to any one of claims 1-8, in association with one or more pharmaceutically acceptable diluents, excipients and/or inert carriers.

10. The pharmaceutical composition according to claim 9, for use in the treatment of VR1 mediated disorders and for treatment of acute and chronic pain disorders, acute and chronic neuropathic pain and acute and chronic inflammatory pain, and respiratory diseases.

11. The compound according to any one of claims 1-8, for use in therapy.

12. Use of a compounds according to any one of claims 1-8, in the manufacture of a medicament for treatment of VR1 mediated disorders.

13. The use according to claim 12 for treatment of acute and chronic pain disorders.

14. The use according to claim 12 for treatment of acute and chronic neuropathic pain.

15. The use according to claim 12 for treatment of acute and chronic inflammatory pain.

16. The use according to claim 12 for treatment of low back pain, post-operative pain, visceral pains like chronic pelvic pain, cystitis, including interstitial cystitis and pain related thereto, ischeamic, sciatia, diabetic neuropathy, multiple sclerosis, arthritis, fibromyalgia, psoriasis, cancer, emesis, urinary incontinence, hyperactive bladder, HIV neuropathy, gastro-esophageal reflux disease (GERD), irritable bowel syndrome (IBS), inflammatory bowel disease (IBD) and pancreatitis.

17. The use according to claim 12 for treatment of respiratory diseases.

18. A method of treatment of VR1 mediated disorders and for treatment of acute and chronic pain disorders, acute and chronic neuropathic pain and acute and chronic inflammatory pain, and respiratory diseases, comprising administering to a mammal, including man in need of such treatment, a therapeutically effective amount of a compound according to claim 1.

19. A processes for the preparation of the compound of formula I wherein P, R¹, R² and n are defined as in claim 1, comprising:
a) reaction of an aromatic amine of formula II, wherein P′ may suitably be a protecting group such as acetyl, ALLOC or BOC, with a properly substituted acyl chloride III: 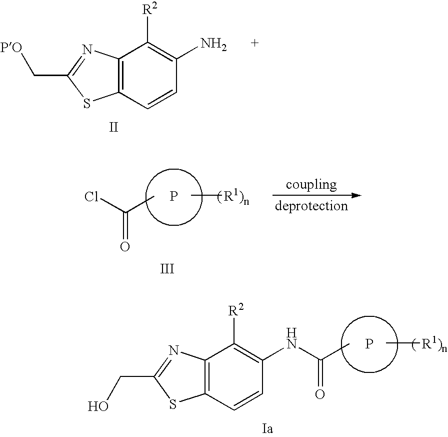
or
b) reaction of an aromatic amine of formula II) wherein P′ may suitably be a protecting group such as acetyl, ALLOC or BOC, with a properly substituted acid IV in the presence of a coupling agent: 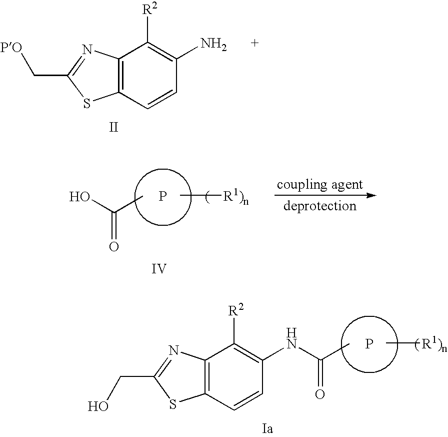
or
c) oxidation of an intermediate Ic to the aldehyde Id 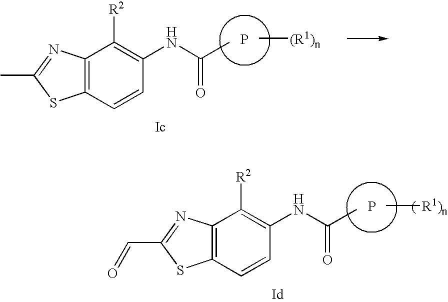
by using a suitable oxidative reagent such as for example, manganese dioxide, chromium trioxide or selenium dioxide;
or
d) reduction of an aldehyde Ie to the alcohol Ib 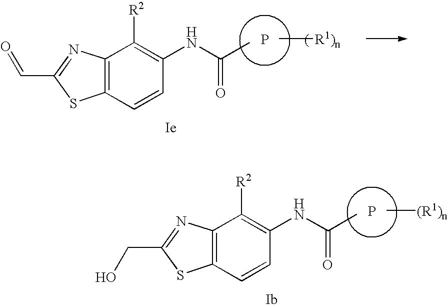
by using a suitable reductive agent such as sodium borohydride;
or,
e) treatment of an aldehyde Ie with organometallic reagent, such as methylmagnesium bromide or methyllithium, leading to an alcohol If 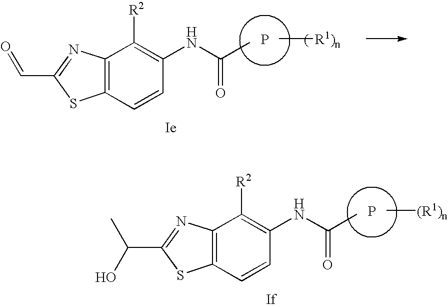
or,
f) oxidation of a 2-methyl derivative Ig and subsequent reduction of the intermediary aldehyde to the 2-hydroxymethyl derivative Ih 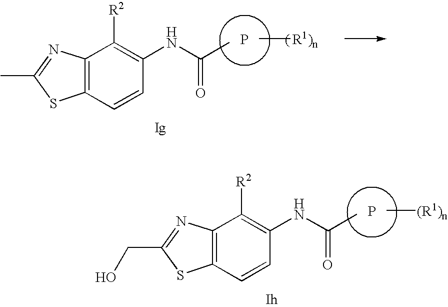
by using a suitable oxidative reagent such as for example, magnesium dioxide, chromium trioxide or selenium dioxide.

20. A compound selected from
4-({[2-({[(allyloxy)carbonyl]oxy}methyl)-1,3-benzothiazol-5-yl]amino}carbonyl)-2,5-dimethylbenzoic acid,
allyl(5-amino-4-chloro-1,3-benzothiazol-2-yl)methyl carbonate,
4-tert-butoxy-2-methylbenzoic acid,
4-isopropoxy-2-methylbenzoic acid,
2-chloro-N-[2-(hydroxymethyl)-1,3-benzothiazol-5-yl]-6-(trifluoromethyl)-nicotinamide,
2-ethyl-4-isopropoxybenzoic acid,
allyl{5-[(tert-butoxycarbonyl)amino]-1,3-benzothiazol-2-yl}methyl carbonate,
allyl{5-[(tert-butoxycarbonyl)amino]-4-fluoro-1,3-benzothiazol-2-yl}methyl carbonate,
allyl(5-amino-4-fluoro-1,3-benzothiazol-2-yl)methyl carbonate,
4-bromo-2-(methoxymethyl)benzoic acid, and 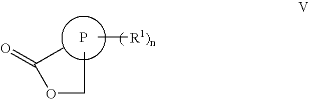
wherein P, R¹ and n are defined as in claim 1.

21. Use of the compounds according to claim 20 as intermediates in the preparation of compounds according to any one of claims 1-8.
